(19) United States
(12) Patent Application Publication
(10) Pub. No.: US 2025/0326849 A1
(43) Pub. Date: Oct. 23, 2025

(54) COMBINATION OF A FcyRIIB- AND A TUMOR ANTIBODY FOR USE IN THE TREATMENT OF AN FcyRIIB-NEGATIVE CANCER

(71) Applicants: UNIVERSITY OF SOUTHAMPTON, Southampton (GB); BIOINVENT INTERNATIONAL AB, Lund (SE)

(72) Inventors: Björn FRENDÉUS, Lund (SE); Linda MÅRTENSSON, Bjärred (SE); Ingrid TEIGE, Lund (SE); Mark CRAGG, Southampton (GB); Robert OLDHAM, Southampton (GB); Stephen BEERS, Southampton (GB); Ali ROGHANIAN, Southampton (GB)

(21) Appl. No.: 18/844,583

(22) PCT Filed: Mar. 6, 2023
(86) PCT No.: PCT/EP2023/055568

§ 371 Date: Sep. 6, 2024

(30) Foreign Application Priority Data
Mar. 7, 2022 (EP) 22160532.2

(51) Int. Cl. C07K 16/28; A61K 39/00; A61P 35/00; C07K 16/32

(52) U.S. Cl. CPC C07K 16/283; A61P 35/00; C07K 16/32; A61K 2039/507; C07K 2317/524; C07K 2317/71

(57) ABSTRACT

Described is the use of a first antibody molecule that specifically binds FcγRIIB via its Fab region, but lacks Fc region or has reduced binding to Fcγ receptors via its Fc region, for use in combination with a second antibody molecule that specifically binds to a receptor present on a tumor cell, which second antibody molecule has an Fc region that binds to at least one activating Fcγ receptor in the treatment of an FcγRIIB-negative cancer in a patient, as well as pharmaceutical compositions and kits including these two antibody molecules, and methods of treating cancer using these two antibodies.

This application is a National Phase entry of International Application No. PCT/EP2023/055568 under § 371 and claims the benefit of European Patent Application No. EP 22160532.2, filed Mar. 7, 2022, which is hereby incorporated by reference in its entirety.

### FIELD OF THE DISCLOSURE

The present disclosure relates to the combined use of 1) an antibody molecule that specifically binds FcγRIIB via its Fab region, and that lacks Fc region or has reduced binding via its Fc region to Fcγ receptors, and 2) an antibody molecule that specifically binds to a receptor present on a tumor cell, which second antibody molecule has an Fc region that binds to at least one activating Fcγ receptor, in treatment of FcγRIIB-negative cancers.

### REFERENCE TO SEQUENCE LISTING

This application contains a Sequence Listing XML that has been submitted electronically in XML format and is hereby incorporated by reference in its entirety. The Sequence Listing was created on Apr. 30, 2025, has a file name of 20250115 SequenceListing APOTT-P002-US.xml, and is 172 kilobytes in size.

### BACKGROUND

It has long been appreciated that the inhibitory Fc gamma receptor (FcγR) IIB, expressed by numerous cells of the immune system, negatively regulates both innate and adaptive immunity through engagement of immune complexes (IC). Similarly, the knowledge that FcγRIIB negatively regulates monoclonal antibody mediated immunotherapy has been known for over a decade. As such, FcγRIIB-deficient mice are able to clear tumors more effectively than wild type (WT) mice when treated with therapeutic monoclonal antibodies (mAbs), indicating that FcγRIIB expression on effector cells (i.e., macrophages and monocytes) leads to suppression of their phagocytic and cytotoxic potential in vivo. Moreover, FcγRIIB regulates the antigen-presenting potential of dendritic cells (DC), and FcγRIIB negative DCs have an improved capacity to activate naive T cells (van Montfoor et al., J Immunol. 2012 Jul. 1; 189(1):92-101). Recently, antagonist antibodies that block FcγRIIB-signalling and internalization in B cells were developed. Such antibodies showed efficient deletion of FcγRIIB-expressing B cells, and efficiently boosted rituximab-mediated deletion of normal and malignant B cells, demonstrating a utility in hematologic cancer (WO 2012/022985). FcγRIIB-blocking antibodies with wildtype IgG1 Fc-proficient in FcγR-binding function, and FcγRIIB-blocking antibodies with an Fc engineered for impaired FcγR-binding (IgG1 N297Q) showed similar ability to enhance rituximab-mediated B cell depletion, indicating that rituximab boosting effects were anti-FcγRIIB Fc-independent. It was, however, not examined or demonstrated whether such antibodies would have utility also in enhancing therapeutic activity of tumor direct-targeting antibodies, e.g., anti-HER2 or anti-EGFR, in treatment of FcγRIIB negative cancers, such as most solid cancers.

Recently, we demonstrated differential antitumor enhancing effects of Fc-FcγR proficient and impaired anti-FcγRIIB antibodies on therapeutic activity of immune modulatory antibodies to the T cell expressed immune inhibitory checkpoints CTLA-4 and PD-1. Specifically, in the context of anti-CTLA-4 the strongest antitumor enhancing effects were observed with Fc-FcγR-impaired anti-FcγRIIB antibodies (WO 2019/138005). Conversely, in the context of combination immunotherapy with anti-PD-1 antibodies Fc-proficient, but not Fc-impaired, anti-FcγRIIB enhanced therapeutic antitumor activity (WO 2021/009358). While studies in genetic knock-out animals had indicated a potential therapy enhancing effect for anti-FcγRIIB antibodies with tumor-direct targeting antibodies both in FcγRIIB+ (e.g. B cell lymphoma when combined with anti-CD20 antibodies) and FcγRIIB− cancers (e.g. solid cancers in combination with anti-HER2 antibodies) (Clynes et al, Nat Med. 2000 April; 6(4):443-6), it remained unclear whether, and if so what type of, anti-FcγRIIB antibodies would enhance therapeutic activity of tumor direct-targeting antibodies e.g. anti-HER2 in treatment of FcγRIIB− cancers.

### SUMMARY

Herein, we demonstrate that only anti-FcγRIIB antibodies lacking Fc region, or whose Fc-region shows reduced or impaired binding to FcγRs e.g. F(ab)′₂ antibodies or aglycosylated antibodies, are able to enhance the therapeutic activity of tumor direct-targeting antibodies e.g. anti-HER2 and anti-EGFR used for treatment of FcγRIIB-negative cancers, including solid cancers. This contrasts to our previous patent applications describing broad use of Fc:FcγR-proficient as well as Fc:FcγR-impaired anti-FcγRIIB antibodies in boosting activity and overcoming resistance to B-cell direct-targeting antibodies, e.g. anti-CD20 for therapy of NHL (WO 2012/022985), and the differential Fc:FcγR-dependence of anti-FcγRIIB to enhance therapeutic activity of immune modulatory (as opposed to tumor cell direct-targeting) anti-PD-1 and anti-CTLA-4 antibodies described in patent applications WO 2021/009358 and WO 2019/138005. Moreover, our data demonstrate that combined treatment with anti-FcγRIIB antibodies lacking Fc region, or whose Fc-region shows reduced or impaired binding to FcγRs, e.g.

F(ab)′₂ antibodies or aglycosylated antibodies, enable anti-HER2 treatment of cancers having a low expression of HER2, which are not indicated for treatment with currently used, clinically approved anti-HER2 regimens.

Disclosed herein is a first antibody molecule that specifically binds FcγRIIB via (or through) its Fab region and that lacks Fc region or has reduced binding to Fcγ receptors via (or through) its Fc region, for use in combination with
a second antibody molecule that specifically binds to a receptor present on a tumor cell, which second antibody molecule has an Fc region that binds to at least one activating Fcγ receptor;
in the treatment of an FcγRIIB-negative cancer in a patient.

Disclosed herein is also a pharmaceutical composition comprising:
(i) a first antibody molecule that specifically binds FcγRIIB via its Fab region and that lacks Fc region or has reduced binding to Fcγ receptors via its Fc region, and
(ii) a second antibody molecule that specifically binds to a receptor present on a tumor cell, which second antibody molecule has an Fc region that binds to at least one activating Fcγ receptor;
for use in the treatment of an FcγRIIB-negative cancer in a patient.

Disclosed herein is further a kit for use in the treatment of an FcγRIIB-negative cancer comprising:
(i) a first antibody molecule that specifically binds FcγRIIB via its Fab region and that lacks Fc region or has a reduced binding to Fcγ receptors via its Fc region, and
(ii) a second antibody molecule that specifically binds to a receptor present on a tumor cell, which second antibody molecule has an Fc region that binds to at least one activating Fcγ receptor.

Further disclosed herein is the use of:
(i) a first antibody molecule that specifically binds FcγRIIB via its Fab region and that lacks Fc region or has reduced binding to Fcγ receptors via its Fc region, and
(ii) a second antibody molecule that specifically binds to a receptor present on a tumor cell, which second antibody molecule has an Fc region that binds to at least one activating Fcγ receptor;
in the manufacture of a medicament for use in the treatment of an FcγRIIB-negative cancer in a patient.

Disclosed herein is also a method for treatment of an FcγRIIB-negative cancer in a patient, comprising administering:
(i) a first antibody molecule that specifically binds FcγRIIB via its Fab region and that lacks Fc region or has reduced binding to Fcγ receptors via its Fc region, and
(ii) a second antibody molecule that specifically binds to a receptor present on a tumor cell, which second antibody molecule has an Fc region that is capable of activating at least one activating Fcγ receptor.

### DETAILED DESCRIPTION

Thus, the present disclosure concerns the combined use of:
(i) a first antibody molecule that specifically binds FcγRIIB via its Fab region and that lacks Fc region or has reduced binding to Fcγ receptors via its Fc region, and
(ii) a second antibody molecule that specifically binds to a receptor present on a tumor cell, which second antibody molecule has an Fc region that is capable of activating at least one activating Fcγ receptor.

The second antibody molecule is thus a tumor direct-targeting antibody or, as it is also called, a direct tumor targeting antibody. The therapeutic activity of this antibody is dependent on engagement of FcγRs. The binding of the second antibody molecule to the receptor on the tumor cell and subsequent engagement of FcγR on an immune effector cell, triggers re-directed FcγR-dependent immune effector cell-mediated killing of the antibody-coated targeted tumor cell, e.g. by macrophage-dependent ADCC or ADCP. The tumor direct-targeting antibody may or may not afford tumor cell killing by additional mechanisms, e.g., by blockade of tumor growth factor signalling, as is thought to be the case for certain anti-HER2 antibodies. Regardless, the present disclosure is applicable to any tumor direct-targeting antibody, whose mechanism encompasses FcγR-dependent tumor cell killing. As such the present disclosure is about maximizing therapeutic activity by optimizing FcγR-dependent tumor cell-killing.

This combination is intended to be used in the treatment of an FcγRIIB-negative cancer in a patient, with the aim to improve therapeutic efficacy of the second antibody molecule through enhanced binding of its Fc part to activatory FcγRs, with reduced binding/activation of inhibitory FcγR.

Fc receptors are membrane proteins which are found on the cell surface of immune effector cells, such as macrophages. The name is derived from their binding specificity for the Fc region of antibodies, which is the usual way an antibody binds to the receptor. However, certain antibodies can also bind the Fc receptors via the antibodies' CDR sequences in the case of antibodies specifically binding to one or more Fc receptors.

A subgroup of the Fc receptors are Fcγ receptors (Fc-gamma receptors, FcgammaR, FcgR), which are specific for IgG antibodies. There are two types of Fcγ receptors: activating Fcγ receptors (also denoted activatory Fcγ receptors) and inhibitory Fcγ receptors. The activating and the inhibitory receptors transmit their signals via immunoreceptor tyrosine-based activation motifs (ITAM) or immunoreceptor tyrosine-based inhibitory motifs (ITIM), respectively. In humans, FcγRIIB (FcγRIIb, FcgRIIB, CD32b) is an inhibitory Fcγ receptor, while FcγRI (CD64), FcγRIIA (CD32a), FcγRIIC (CD32c), FcγRIIIA (CD16a) and FcγRIV are activating Fcγ receptors. FcγgRIIIB is a GPI-linked receptor expressed on neutrophils that lacks an ITAM motif but through its ability to cross-link lipid rafts and engage with other receptors is also considered activatory. In mice, the activating receptors are FcγRI, FcγRIII and FcγRIV.

It is well-known that antibodies modulate immune cell activity through interaction with Fcγ receptors. Specifically, how antibody immune complexes modulates immune cell activation is determined by their relative engagement of activating and inhibitory Fcγ receptors. Different antibody isotypes bind with different affinity to activating and inhibitory Fcγ receptors, resulting in different A:I ratios (activation:inhibition ratios) (Nimmerjahn et al; Science. 2005 Dec. 2; 310(5753):1510-2).

By binding to an inhibitory Fcγ receptor, an antibody can inhibit, block and/or downmodulate effector cell functions.

By binding to an activating Fcγ receptor, an antibody can activate effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

The antibody molecule according to at least one embodiment of the invention specifically binds FcγRIIB, i.e. the first antibody, binds to or interacts with this Fcγ receptor via the Fab region of the antibody, i.e. via the antigen-binding region on an antibody that binds to antigens which is composed of one constant and one variable domain of each of the heavy and the light chain. In particular, it binds to FcγRIIB present on an immune effector cell, and in particular to FcγRIIB present on the surface of an immune effector cell. If this antibody would have had a usual or ordinary Fc region, the antibody could also have bound to an activating Fcγ receptor through normal interaction between the Fc region and Fc receptor. However, in at least one embodiment of the invention, the antibody molecule that specifically binds FcγRIIB completely lacks Fc region or has reduced binding to Fcγ receptors, which means that the antibody molecule that specifically binds FcγRIIB binds poorly to or cannot at all bind to or interact with Fcγ receptors. This appears to have at least two therapeutically important consequences:
1) lack of Fc-mediated binding to activatory FcγRs leaves a greater number of activatory Fc gamma receptors available for binding to Fc's of (other) therapeutic anti-cancer antibodies. This is important since clustering of an increasing number of activatory FcγRs (vs inhibitory FcγRs; Nimmerjahn et al; Science. 2005 Dec. 2; 310(5753):1510-2) is known to increase effector cell mediated target cell deletion, a mechanism underlying activity of both checkpoint inhibitor, immune agonist, and other immunomodulatory antibodies, such as anti-IL-2R.
2) lack of, or reduced, Fc-mediated binding to inhibitory FcγR was shown to reduce inhibitory signalling in FcγR-expressing immune effector cells. Thus, lack of or reduced Fc-mediated binding to FcγR of the FcγRIIB targeting antibody likely improves therapeutic efficacy by at least two mechanisms, involving both improved activatory FcγR and reduced inhibitory Fcγ signalling in immune effector cells in response to a second immunomodulatory anti-cancer antibody.

“Reduced binding” or “binding with reduced affinity” means in this context that antibody molecule has reduced Fc mediated binding to Fcγ receptors, or in other words that the Fc region of the antibody molecule that specifically binds FcγRIIB binds to an activating Fcγ receptor with lower affinity than the Fc region of a normal human IgG1. The reduction in binding can be assessed using techniques such as surface plasmon resonance. In this context “normal IgG1” means a conventionally produced IgG1 with a non-mutated Fc region that has not been produced so as to alter its glycosylation. As a reference for this “normal IgG1” it is possible to use rituximab produced in CHO cells without any modifications (Tipton et al, Blood 2015 125:1901-1909; rituximab is described e.g., in EP 0 605 442).

“Reduced binding” means that binding of the Fc region of the antibody molecule that specifically binds FcγRIIB binds to an activating Fcγ receptor is at least 10-fold reduced for all Fc receptors compared to the binding of the Fc region of a normal human IgG1 to the same receptors. In some embodiments it is at least 20-fold reduced. In some embodiments it is at least 30-fold reduced. In some embodiments it is at least 40-fold reduced. In some embodiments it is at least 50-fold reduced. In some embodiments it is at least 60-fold reduced. In some embodiments it is at least 70-fold reduced.

In some embodiments of the present invention, the antibody molecule that specifically binds FcγRIIB does not bind at all with its Fc region, and in some such cases the antibody does not have an Fc region; it may then be a Fab, Fab′₂, scFv or PEGYLATED versions thereof.

In some embodiments, the antibody molecule that specifically binds FcγRIIB may be a llama antibody, and in particular a llama hcIgG. Like all mammals, camelids produce conventional antibodies made of two heavy chains and two light chains bound together with disulphide bonds in a Y shape (IgG₁). However, they also produce two unique subclasses of immunoglobulin G, IgG₂ and IgG₃, also known as heavy chain IgG (hcIgG). These antibodies are made of only two heavy chains that lack the CH1 region but still bear an antigen binding domain at their N-terminus called V_{H}H. Conventional Ig requires the association of variable regions from both heavy and light chains to allow a high diversity of antigen-antibody interactions. Although isolated heavy and light chains still show this capacity, they exhibit very low affinity when compared to paired heavy and light chains. The unique feature of hcIgG is the capacity of their monomeric antigen binding regions to bind antigens with specificity, affinity and especially diversity that are comparable to conventional antibodies without the need of pairing with another region.

In some embodiments reduced binding means that the antibody has a 20-fold reduced affinity with regards to binding to FcγRI.

In order to obtain reduced binding of an IgG1 antibody, such as an IgG1 antibody, to an Fc receptor, it is possible to modify the Fc region of the IgG antibody by aglycosylation. Such aglycosylation, for example of an IgG1 antibody, may for example be achieved by an amino acid substitution of the asparagine in position 297 (N297X) in the antibody chain. The substation may be with a glutamine (N297Q), or with an alanine (N297A), or with a glycine (N297G), or with an asparagine (N297D), or by a serine (N297S).

The Fc region may be modified by further substitutions, for example as described by Jacobsen F W et al., JBC 2017, 292, 1865-1875, (see e.g. Table 1). Such additional substitutions include L242C, V259C, A287C, R292C, V302C, L306C, V323C, 1332C, and/or K334C. Such modifications also include the following combinations of substitutions in an IgG1:
L242C, N297G, K334C,
A287C, N297G, L306C,
R292C, N297G, V302C,
N297G, V323C, 1332C, and
V259C, N297G, L306C.

Alternatively, the carbohydrate in the Fc region can be cleaved enzymatically and/or the cells used for producing the antibody can be grown in media that impairs carbohydrate addition and/or cells engineered to lack the ability to add the sugars can be used for the antibody production, or by production of antibodies in host cells that do not glycosylate or do not functionally glycosylate antibodies e.g. prokaryotes including E. coli, as explained above.

Reduced affinity for Fc gamma receptors can further be achieved through engineering of amino acids in the antibody Fc region (such modifications have previously been described by e.g. Xencor, Macrogenics, and Genentech), or by production of antibodies in host cells that do not glycosylate or does not functionally glycosylate antibodies e.g. prokaryotes including E. coli.

In addition to having reduced binding to Fcγ receptors through the Fc region, it is in some embodiments preferred that the antibody molecule that specifically binds FcγRIIB does not give rise to phosphorylation of FcγRIIB when binding the target. Phosphorylation of the ITIM of FcγRIIB is an inhibitory event that blocks the activity in the immune cell.

Fc gamma receptor expressing immune effector cell refers herein to principally innate effector cells, and includes specifically macrophages, neutrophils, monocytes, natural killer (NK) cells, basophils, eiosinophils, mast cells, and platelets. Cytotoxic T cells and memory T cells do not typically express FcγRs, but may do so in specific circumstances. In some embodiments the immune effector cell is an innate immune effector cell. In some embodiments, the immune effector cell is a macrophage.

Contrary to the antibody molecule that specifically binds FcγRIIB, the antibody molecule that specifically binds to or interacts with a receptor present on a tumor cell, i.e. the second antibody molecule, or the tumor direct-targeting antibody, has an Fc region that binds to or interacts with an activating Fcγ receptor in an extent that is not reduced or at least not substantially reduced. The binding of the second antibody to the tumor cell results in activation of Fc receptor dependent anti-tumor activity, such as depletion, antibody-dependent cellular cytotoxicity (ADCC) and/or antibody-dependent cellular phagocytosis (ADCP). By depletion, we refer herein to depletion, deletion or elimination of tumor cells through physical clearance of cells causes depletion of that tumor cell.

To decide whether an antibody molecule is a tumor depleting antibody molecule in the meaning of the present disclosure, it is possible to use an in vitro ADCC or ADCP assay. To decide whether an antibody molecule is a tumor cell depleting antibody molecule the same assay would be performed in the presence of and without the depleting antibody, which would show whether or not the depleting antibody to be tested is in fact depleting.

An ADCC assay may be done by labelling target cells with calcein AM (acetyl methyl ester), followed by the addition of diluting concentrations of antibody. Target cells is then cocultured with human peripheral blood mononuclear cells (PBMCs) at a 50:1 effector: target (E:T) ratio for 4 h at 37° C. The plate is centrifuged at 400×g for 5 min to pellet the cells, and the supernatant is transferred to a white 96-well plate. Calcein release is measured using a Varioskan (Thermo Scientific) using an excitation wavelength of 485 nm and emission wavelength, 530 nm. The percentage of maximal release is calculated as follows: % max release=(sample/triton treated)*100.

An ADCP assay may be done by labelling target cells with 5 mM carboxyfluorescein succinimidyl ester (CFSE) for 10 min at room temperature before washing in media containing fetal calf serum. CFSE-labelled targets is then opsonized with diluting concentrations of antibody before coculturing at a 1:5 E:T ratio with bone marrow derived macrophages (BMDMs) in 96-well plates for 1 h at 37° C. BMDMs are then labelled with anti-F4/80-allophycocyanin for 15 min at room temperature and washed with PBS twice. Plates are kept on ice, wells are scraped to collect BMDMs, and phagocytosis is assessed by flow cytometry using a FACSCalibur (BD) to determine the percentage of F4/80+CFSE+ cells within the F4/80+ cell population.

It is also possible to use a method as described by Cleary et al in J Immunol, Apr. 12, 2017, 1601473.

The tumor cell to which the second antibody molecule binds is a FcγRIIB-negative cancer tumor, which means that it is a tumor that does not present any FcγRIIB receptors. This can be tested using anti-FcγRIIB specific antibodies in a variety of methods including immunohistochemistry and flow cytometry such as indicated in Tutt et al J Immunol 2015, 195 (11) 5503-5516.

In addition to binding specifically to a target on the tumor cell, the second antibody molecule binds via its Fc region to an activating Fcγ receptor present on an immune effector cell. In order to be able to bind to an activating Fcγ receptor, the Fc region of the second antibody should at least in some embodiments be glycosylated at position 297. The carbohydrate residue in this position helps binding to Fcγ receptors. In some embodiments it is preferred that these residues are biantennary carbohydrates which contain GlnNAc, mannose, with terminal galactose residues and sialic acid. It should contain the CH₂ part of the Fc molecule.

Antibodies are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fv region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2.

Another part of an antibody is the Fc region (otherwise known as the fragment crystallizable domain), which comprises two of the constant domains of each of the antibody's heavy chains. As mentioned above, the Fc region is responsible for interactions between the antibody and Fc receptor.

The term antibody molecule, as used herein, encompasses full-length or full-size antibodies as well as functional fragments of full length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted F(ab), which is a monovalent antigen-binding fragment that does not contain a Fc part, or a F(ab′)₂ (also denoted Fab′₂ or Fab₂), which is an divalent antigen-binding fragment that contains two antigen-binding Fab parts linked together by disulfide bonds, or a F(ab′), i.e. a monovalent-variant of a F(ab′)₂. Such a fragment may also be single chain variable fragment (scFv).

A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269: 32389-93 it is described that a whole VL chain (including all three CDRs) has a high affinity for its substrate.

Molecules containing two CDR regions are described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362: 293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272: 30937-44, in which it is demonstrated that a range of hexapeptides derived from a CDR display antigen-binding activity and it is noted that synthetic peptides of a complete, single, CDR display strong binding activity. In Monnet et al., 1999, JBC, 274: 3789-96 it is shown that a range of 12-mer peptides and associated framework regions have antigen-binding activity and it is commented on that a CDR3-like peptide alone is capable of binding antigen. In Heap et al., 2005, J. Gen. Virol., 86: 1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. When a derivative is used it should have the same antigen binding characteristics as the corresponding full-length antibody in the sense that it binds to the same epitope on the target as the full-length antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, antibodies of human origin, humanized antibodies, chimeric antibodies, single chain antibodies, single-chain Fvs (scFv), Fab fragments, F(ab′)₂ fragments, F(ab′) fragments, disulfide-linked Fvs (sdFv), antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE, unless otherwise specified.

In some embodiments, the antibody is a human IgG1. The skilled person will appreciate that the mouse IgG2a and human IgG1 engage with activatory Fc gamma receptors, and share the ability to activate deletion of target cells through activation of activatory Fc gamma receptor bearing immune cells by e.g. ADCP and ADCC. As such, in embodiments where the mouse IgG2a is the preferred isotype for deletion in the mouse, human IgG1 is a preferred isotype for deletion in human in such embodiments.

As outlined above, different types and forms of antibody molecules are encompassed by at least one embodiment of the invention, and would be known to the person skilled in immunology. It is well known that antibodies used for therapeutic purposes are often modified with additional components which modify the properties of the antibody molecule.

Accordingly, we include that an antibody molecule of at least one embodiment of the invention or an antibody molecule used in accordance with at least one embodiment of the invention (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualized in vitro, and/or in vivo, and/or ex vivo.

By “cytotoxic moiety”, we include a radioactive moiety, and/or enzyme, wherein the enzyme is a caspase, and/or toxin, wherein the toxin is a bacterial toxin or a venom; wherein the cytotoxic moiety is capable of inducing cell lysis.

We further include that the antibody molecule may be in an isolated form and/or purified form, and/or may be PEGylated. PEGylation is a method by which polyethylene glycol polymers are added to a molecule such as an antibody molecule or derivative to modify its behavior, for example to extend its half-life by increasing its hydrodynamic size, preventing renal clearance.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT® (http://www.imgt.org/and Lefranc and Lefranc “The Immunoglobulin FactsBook” published by Academic Press, 2001).

The antibody molecule of at least a further embodiment of the present invention or used according to at least a further embodiment of the invention is an antibody molecule that is capable of competing with the specific antibodies provided herein, for example antibody molecules comprising any of the amino acid sequences set out in for example SEQ ID NOs: 1-194 for binding to the specific target.

By “capable of competing for” we mean that the competing antibody is capable of inhibiting or otherwise interfering, at least in part, with the binding of an antibody molecule as defined herein to the specific target.

For example, such a competing antibody molecule may be capable of inhibiting the binding of an antibody molecule described herein by at least about 10%; for example at least about 20%, or at least about 30%, at least about 40%, at least about 50%, at least about 60%, at least about 70%, at least about 80%, at least about 90%, at least about 95%, about 100% and/or inhibiting the ability of the antibody described herein to prevent or reduce binding to the specific target by at least about 10%; for example at least about 20%, at least about 30%, at least about 40%, at least about 50%, at least about 60%, at least about 70%, at least about 80%, at least about 90%, at least about 95%, or about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

The targets of the antibodies according to at least one embodiment the present invention, or of the antibodies used in accordance with at least one embodiment of the invention, are expressed on the surface of cells, i.e. they are cell surface antigen, which would include an epitope (otherwise known in this context as a cell surface epitope) for the antibody. Cell surface antigen and epitope are terms that would be readily understood by one skilled in immunology or cell biology.

By “cell surface antigen”, we include that the cell surface antigen is exposed on the extracellular side of the cell membrane, but may only be transiently exposed on the extracellular side of the cell membrane. By “transiently exposed”, we include that the cell surface antigen may be internalized into the cell, or released from the extracellular side of the cell membrane into the extracellular space. The cell surface antigen may be released from the extracellular side of the cell membrane by cleavage, which may be mediated by a protease.

We also include that the cell surface antigen may be connected to the cell membrane, but may only be transiently associated with the cell membrane. By “transiently associated”, we include that the cell surface antigen may be released from the extracellular side of the cell membrane into the extracellular space. The cell surface antigen may be released from the extracellular side of the cell membrane by cleavage, which may be mediated by a protease.

We further include that the cell surface antigen may be a peptide, or a polypeptide, or a carbohydrate, or an oligosaccharide chain, or a lipid; and/or an epitope that is present on a protein, or a glycoprotein, or a lipoprotein.

Methods of assessing protein binding are known to the person skilled in biochemistry and immunology. It would be appreciated by the skilled person that those methods could be used to assess binding of an antibody to a target and/or binding of the Fc region of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

What is meant by an antibody that specifically binds to or interacts with a defined target molecule or antigen is well known, and means that the antibody preferentially and selectively binds its target and not a molecule which is not a target. In this context, the term “binds to” can be used interchangeably with “interacts with”. Accordingly, by “antibody molecule the specifically binds” or “target specific antibody molecule” we include that the antibody molecule specifically binds a target but does not bind to non-target, or binds to a non-target more weakly (such as with a lower affinity) than the target.

We also include the meaning that the antibody specifically binds to the target at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target if it binds to the target with a K_{d} of at least about 10⁻¹ K_{d}, or at least about 10⁻² K_{d}, or at least about 10⁻³ K_{d}, or at least about 10⁻⁴ K_{d}, or at least about 10⁻⁵ K_{d}, or at least about 10⁻⁶ K_{d}, or at least about 10⁻⁷ K_{d}, or at least about 10⁻⁸ K_{d}, or at least about 10⁻⁹ K_{d}, or at least about 10⁻¹⁰ K_{d}, or at least about 10⁻¹¹ K_{d}, or at least about 10⁻¹² K_{d}, or at least about 10⁻¹³ K_{d}, or at least about 10⁻¹⁴ K_{d}, or at least about 10⁻¹⁵ K_{d}.

In some embodiments the antibody molecule that specifically binds FcγRIIB is a human antibody.

In some embodiments, the antibody molecule that specifically binds FcγRIIB is an antibody of human origin, i.e. an originally human antibody that has been modified as described herein.

In some embodiments, the antibody molecule that specifically binds FcγRIIB is a humanized antibody, i.e. an originally non-human antibody that has been modified to increase its similarity to a human antibody. The humanized antibodies may, for example, be of murine antibodies or llama antibodies.

In some embodiments, the antibody molecule that specifically binds FcγRIIB comprises the following constant regions (CH and CL):

IgG1-CH
[SEQ ID NO: 1]
ASTKGPSVFPLAPSSKSTSGGTAALGCLVKDYFPEPVTVSWNSGA
LTSGVHT-FPAVL-QSSGLYSLSSVVTVPSSSLGTQTYICNVNHK
PSNTKVDKKVE-PKSCDKTHTCPPCP-APELLGGPSVFLFPPKPK
DTLMISRTPEVTCVVVDVSHEDPEVKFNWY-VDGVEVHNAK-TKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPEN-NYKTTPPVLDSDGS-FFLYSKLTVDKSRWQQGNVFS
CSVMHEALHNHYTQKSLSLSPGK
IgG1-CL
[SEQ ID NO: 2]
QPKAAPSVTLFPPSSEELQANKATLVCLISDFYPGAVTVAWKADS
SPV-KAGVETTTPSKQSNNKYAASSYLSLTPEQWKSHRSYSCQVT
HEGSTVEKTVAPTECS

These constant regions (SEQ ID NO: 1 and SEQ ID NO: 2) are of human origin. The Fc region is further modified for reduced binding to Fcγ receptors via its Fc region. As mentioned herein, it is in some embodiments preferred that SEQ ID NO: 1 has been aglycosylated through an N297Q substitution, and the IgG1-CH has then the following CH sequence [SEQ ID NO: 195], with the 297 Q residue is marked in bold:

ASTKGPSVFPLAPSSKSTSGGTAALGCLVKDYFPEPVTVSWNSGA
LTSGVHT-FPAVLQSSGLYSLSSVVTVPSSSLGTQTYICNVNHKP
SNTKVDKKVEPKSCDKTHTCPPCPAPELLGGPSVFLFPPKPKDTL
MIS-RTPEVTCVVVDVSHEDPEVKFNWY-VDGVEVHNAKTKPREE
QYQSTYRVVSVLTVLHQDWLNGKEYKCKVSNKALPAPIEKTISKA
KGQPREPQVYTLPPSRDELTKNQVSLTCLVKGFYPSDI-AVEWES
NGQPEN-NYKTTPPVLDSDGSFFLYSKLTVDKSRWQQGNVFSCSV
MHEALHNHYTQKSLSLSPGK

In some embodiments and/or examples, murine antibody molecules are used. These may also be used for surrogate antibodies. These may then comprise the following constant regions (CH and CL):

CH
[SEQ ID NO: 196]
AKTTAPSVYPLAPVCGDTTGSSVTLGCLVKGYFPEPVTLTWNSGS
LSSGVHT-FPAVLQSDLYTLSSSVTVTSSTWPSQSITCNVAHPAS
STKVDKKIEPRGPTIKP-CPPCKCPAPNLLGGPSVFIFPPKIKDV
LMIS-LSPIVTCVVVDVSEDDPDVQISWFVNN-VEVHTAQTQTHR
EDYASTLRVVSALPIQHQDWMSGKEFKCKVNNKDLPA-PIERTI-
SKPKGSVRAPQVYVLPPPEEEMTKKQVTLTCMVTDFMPEDIYVEW
TNNGKTEL-NY-KNTEPVLDSDGSYFMYSKLRVEKKNWVERNSYS
CSVVHEGLHNHHTTKS-FSRTPGK
CL
[SEQ ID NO: 197]
QPKSSPSVTLFPPSSEELETNKA-TLVCTITDFYPGVVTVDWKV
DGTPVTQGMET-TQPSKQSNNKYMASSYLTLTARAWERHSSYSC
QVTHEGHTVEKSLSRADCS

These constant regions (SEQ ID NO: 196 and SEQ ID NO: 197) are thus of murine origin. SEQ ID NO: 196 comprises the N297A mutation (the 297 A residue is marked in bold in the sequence above). This N297A mutation in the murine sequence corresponds to the N297Q mutation in the human sequence.

In some embodiments, the antibody molecule that specifically binds FcγRIIB comprises one or more sequences of the following clones:

Antibody clone: 1A01
1A01-VH
[SEQ ID NO: 3]
EVQLLESGGGLVQPGGSLRLSCAASGFTFSDYYMNWIRQ
TPGKGLEWVSLIG-WDGGSTY-YADSVKGRFTISRDN
SENTLYLQMNSLRAEDTAVYYCARAYSGYELDYWGQGT
LVTVSS
1A01-VL
[SEQ ID NO: 27]
QSVLTQPPSASGTPGQRVTISCSGSSSNIGNNAVNWYQQLPGTAPKLLI-
YDNNNRPSGVP-
DRFSGSKSGTSASLAISGLRSEDEADYYCAAWDDSLNASIFGGGTKLTVLG
CDR regions
CDRH1:
[SEQ ID NO: 51]
DYYMN
CDRH2:
[SEQ ID NO: 52]
LIGWDGGSTYYADSVKG
CDRH3:
[SEQ ID NO: 53]
AYSGYELDY
CDRL1:
[SEQ ID NO: 54]
SGSSSNIGNNAVN
CDRL2:
[SEQ ID NO: 55]
DNNNRPS
CDRL3:
[SEQ ID NO: 56]
AAWDDSLNASI
Antibody clone: 1B07
1B07-VH
[SEQ ID NO: 4]
EVQLLESGGGLVQPGGSLRLSCAASGFTFSSYGMHWVR-
QAPGKGLEWVAFTRYDGSNKY-YADSVRGRFTISRDNSKN
TLYLQMNSLRAEDTAVYYCARENIDAFDVWGQGTL
VTVSS
1B07-VL
[SEQ ID NO: 28]
QSVLTQPPSASGTPGQRVTISCSGSSSNIGNNAVNWYQQLPGTAPKLLI-
YDNQQRPSGVP-
DRFSGSKSGTSASLAISGLRSEDEADYYCEAWDDRLFGPVFGGGTKLTVLG
CDR regions
CDRH1:
[SEQ ID NO: 57]
SYGMH
CDRH2:
[SEQ ID NO: 58]
FTRYDGSNKYYADSVRG
CDRH3:
[SEQ ID NO: 59]
ENIDAFDV
CDRL1:
[SEQ ID NO: 60]
SGSSSNIGNNAVN
CDRL2:
[SEQ ID NO: 61]
DNQQRPS
CDRL3:
[SEQ ID NO: 62]
WDDRLFGPV
Antibody clone: 1C04
1C04-VH
[SEQ ID NO: 5]
EVQLLESGGGLVQPGGSLRLSCAASGFTFSSYAMSWVR-
QAPGKGLEWVSSISDSGAG-
RYYADSVEGRFTISRDNSKNTLYLQMNSLRAEDTAVYYCAR
THDSGELLDAFDIWGQGTLVTVSS
1C04-VL
[SEQ ID NO: 29]
QSVLTQPPSASGTPGQRVTISCSGSSSNIGSNHVLWYQQLPGTAPKLLI-
YGNSNRPSGVP-
DRFSGSKSGTSASLAISGLRSEDEADYYCAAWDDSLNGWVFGGGTKLTVLG
CDR regions
CDRH1:
[SEQ ID NO: 63]
SYAMS
CDRH2:
[SEQ ID NO: 64]
SISDSGAGRYYADSVEG
CDRH3:
[SEQ ID NO: 65]
THDSGELLDAFDI
CDRL1:
[SEQ ID NO: 66]
SGSSSNIGSNHVL
CDRL2:
[SEQ ID NO: 67]
GNSNRPS
CDRL3:
[SEQ ID NO: 68]
AAWDDSLNGWV
Antibody clone: 1E05
1E05-VH
[SEQ ID NO: 6]
EVQLLESGGGLVQPGGSLRLSCAASGFTFSTY-
AMNWVRQVPGKGLEWVAVISYD-GSNKN
YVDSVKGRFTISRDNSKNTLYLQMNSLRAEDTAVYYCARNFDNSGYAIP
DAFDIWGQGTLVTVSS
1E05-VL
[SEQ ID NO: 30]
QSVLTQPPSASGTPGQRVTISCTGSSSNIGAGYDVHWYQQLPGTAPKLLI-
YDNNSRPSGVP-
DRFSGSKSGTSASLAISGLRSEDEADYYCAAWDDSLGGPVFGGGTKLTVLG
CDR regions
CDRH1:
TYAMN
[SEQ ID NO: 69]
CDRH2:
[SEQ ID NO: 70]
VISYDGSNKNYVDSVKG
CDRH3:
[SEQ ID NO: 71]
NFDNSGYAIPDAFDI
CDRL1:
[SEQ ID NO: 72]
TGSSSNIGAGYDVH
CDRL2:
[SEQ ID NO: 73]
DNNSRPS
CDRL3:
[SEQ ID NO: 74]
AAWDDSLGGPV
Antibody clone: 2A09
2A09-VH
[SEQ ID NO: 7]
EVQLLESGGGLVQPGGSLRLSCAASGFTFSNAWMSWVR-
QAPGKGLEWVAYISRDADITHY-PASVKGRFTISR
DNSKNTLYLQMNSLRAEDTAVYYCTTGFDYAGDDAFDIWGQ
GTLVTVSS
2A09-VL
[SEQ ID NO: 31]
QSVLTQPPSASGTPGQRVTISCSGSSSNIGSNAVNWYQQLPGTAPKLLI-
YGNSDRPSGVP-DRFSGSKSGTSASLAISGLRSEDEADYYC
AAWDDSLNGRWVFGGGTKLTVLG
CDR regions
CDRH1:
[SEQ ID NO: 75]
NAWMS
CDRH2:
[SEQ ID NO: 76]
YISRDADITHYPASVKG
CDRH3:
[SEQ ID NO: 77]
GFDYAGDDAFDI
CDRL1:
[SEQ ID NO: 78]
SGSSSNIGSNAVN
CDRL2:
[SEQ ID NO: 79]
GNSDRPS
CDRL3:
[SEQ ID NO: 80]
AAWDDSLNGRWV
Antibody clone: 2B08
2B08-VH
[SEQ ID NO: 8]
EVQLLESGGGLVQPGGSLRLSCAASGFTFSDYYMSWVR-
QAPGKGLEWVALIGHDGNN-
KYYLDSLEGRFTISRDNSKNTLYLQMNSLRAEDTAVYY
CARATDSGYDLLYWGQGTLVTVSS
2B08-VL
[SEQ ID NO: 32]
QSVLTQPPSASGTPGQRVTISCSGSSSNIGNNAVNWYQQLPGTAP-
KLLIYYDDLLPSGVP-
DRFSGSKSGTSASLAISGLRSEDEADYYCTTWDDSLSGVVFGGGTKLTVLG
CDR regions
CDRH1:
[SEQ ID NO: 81]
DYYMS
CDRH2:
[SEQ ID NO: 82]
LIGHDGNNKYYLDSLEG
CDRH3:
[SEQ ID NO: 83]
ATDSGYDLLY
CDRL1:
[SEQ ID NO: 84]
SGSSSNIGNNAVN
CDRL2:
[SEQ ID NO: 85]
YDDLLPS
CDRL3:
[SEQ ID NO: 86]
TTWDDSLSGVV
Antibody clone: 2E8-VH
2E8-VH
[SEQ ID NO: 9]
EVQLLESGGGLVQPGGSLRLS-
CAASGFTFSDYYMSWIRQAPGKGLEWVSAIGFSDDNTY-
YADSVKGRFTISRDNSKNTLYLQMNSLRAEDTAVYYCAGGDGS
GWSFWGQGTLVTVSS
2E8-VL
[SEQ ID NO: 33]
QSVLTQPPSASGTPGQRVTISCSGSSSNIGNNAVNWYQQLPGTAP
KLLIYDNN-KRPSGVP-DRFSGSKSGTSA
SLAISGLRSEDEADYYCATWDDSLRGWVFGGGTKLTVLG
CDR regions
CDRH1:
[SEQ ID NO: 87]
DYYMS
CDRH2:
[SEQ ID NO: 88]
AIGFSDDNTYYADSVKG
CDRH3:
[SEQ ID NO: 89]
GDGSGWSF
CDRL1:
[SEQ ID NO: 90]
SGSSSNIGNNAVN
CDRL2:
[SEQ ID NO: 91]
DNNKRPS
CDRL3:
[SEQ ID NO: 92]
ATWDDSLRGWV
Antibody clone: 5C04
5C04-VH
[SEQ ID NO: 10]
EVQLLESGGGLVQPGGSLRLSCAASGFTFSNYGMHWVR-
QAPGKGLEWVAVISYDGSNKY-YADSVKGRFTISR
DNSKNTLYLQMNSLRAEDTAVYYCAREWRDAFDIWGQGTL
VTVSS
5C04-VL
[SEQ ID NO: 34]
QSVLTQPPSASGTPGQRVTISCTGSSSNIGAGYDVHWYQQLPGTAP-
KLLIYSDNQRPSGVP-DRFSGSKSGTSASLA
ISGLRSEDEADYYCAAWDDSLSGSWVFGGGTKLTVLG
CDR regions
CDRH1:
[SEQ ID NO: 93]
NYGMH
CDRH2:
[SEQ ID NO: 94]
VISYDGSNKYYADSVKG
CDRH3:
[SEQ ID NO: 95]
WRDAFDI
CDRL1:
[SEQ ID NO: 96]
TGSSSNIGAGYDVH
CDRL2:
[SEQ ID NO: 97]
SDNQRPS
CDRL3:
[SEQ ID NO: 98]
AAWDDSLSGSWV
Antibody clone: 5C05
5C05-VH
[SEQ ID NO: 11]
EVQLLESGGGLVQPGGSLRLSCAASGFTFSTYGMHWVR-
QAPGKGLEWVAVISYDGSNKY-YADSVKGRFTISRDNSK
NTLYLQMNSLRAEDTAVYYCARENFDAFDVWGQGTL
VTVSS
5C05-VL
[SEQ ID NO: 35]
QSVLTQPPSASGTPGQRVTISCTGSSSNIGAGYDVHWYQQLPGTAP-
KLLIYSNSQRPSGVP-DRFSGSKSGTSASLAISGLRSED
EADYYCAAWDDSLNGQVVFGGGTKLTVLG
CDR regions
CDRH1:
[SEQ ID NO: 99]
TYGMH
CDRH2:
[SEQ ID NO: 100]
VISYDGSNKYYADSVKG
CDRH3:
[SEQ ID NO: 101]
ENFDAFDV
CDRL1:
[SEQ ID NO: 102]
TGSSSNIGAGYDVH
CDRL2:
[SEQ ID NO: 103]
SNSQRPS
CDRL3:
[SEQ ID NO: 104]
AAWDDSLNGQVV
Antibody clone: 5D07
5D07-VH
[SEQ ID NO: 12]
EVQLLESGGGLVQPGGSLRLSCAASGFTFSTYGMHWVR-
QAPGKGLEWVAVIAYDGSKKDY-
ADSVKGRFTISRDNSKNTLYLQMNSLRAEDTAVYYCARE
YRDAFDIWGQGTLVTVSS
5D07-VL
[SEQ ID NO: 36]
QSVLTQPPSASGTPGQRVTISCTGSSSNIGAGYDVHWYQQLPGTAPKLLI-
YGNSNRPSGVP-
DRFSGSKSGTTASLAISGLRSEDEADYYCAAWDDSVSGWMFGGGTKLTVLG
CDR regions
CDRH1:
[SEQ ID NO: 105]
TYGMH
CDRH2:
[SEQ ID NO: 106]
VIAYDGSKKDYADSVKG
CDRH3:
[SEQ ID NO: 107]
EYRDAFDI
CDRL1:
[SEQ ID NO: 108]
TGSSSNIGAGYDVH
CDRL2:
[SEQ ID NO: 109]
GNSNRPS
CDRL3:
[SEQ ID NO: 110]
AAWDDSVSGWM
Antibody clone: 5E12
5E12-VH
[SEQ ID NO: 13]
EVQLLESGGGLVQPGGSLRLSCAASGFTFSSYGMHWVR-
QAPGKGLEWVAVISYDGINKDY-
ADSMKGRFTISRDNSKNTLYLQMNSLRAEDTAVYYCARE
RKDAFDIWGQGTLVTVSS
5E12-VL
[SEQ ID NO: 37]
QSVLTQPPSASGTPGQRVTISCTGSSSNIGAGYDVHWYQQLPGTAP-
KLLIYSNNQRPSGVP-DRFSGSKSGT
SASLAISGLRSEDEADYYCATWDDSLNGLVFGGGTKLTVLG
CDR regions
CDRH1:
[SEQ ID NO: 111]
SYGMH
CDRH2:
[SEQ ID NO: 112]
VISYDGINKDYADSMKG
CDRH3:
[SEQ ID NO: 113]
ERKDAFDI
CDRL1:
[SEQ ID NO: 114]
TGSSSNIGAGYDVH
CDRL2:
[SEQ ID NO: 115]
SNNQRPS
CDRL3:
[SEQ ID NO: 116]
ATWDDSLNGLV
Antibody clone: 5G08
5G08-VH
[SEQ ID NO: 14]
EVQLLESGGGLVQPGGSLRLSCAASGFTFNNYGMHWVR-
QAPGKGLEWVAVISYDGSN-
RYYADSVKGRFTMSRDNSKNTLYLQMNSLRAEDTAVYYCARDRWNGMDVWG
QGTLVTVSS
5G08-VL
[SEQ ID NO: 38]
QSVLTQPPSASGTPGQRVTISCSGSSSNIGAGYDVHWYQQLPGTAPKLLI-
YANNQRPSGVP-DRFSGSKSG
TSASLAISGLRSEDEADYYCAAWDDSLNGPWVFGGGTKLTVLG
CDR regions
CDRH1:
[SEQ ID NO: 117]
NYGMH
CDRH2:
[SEQ ID NO: 118]
VISYDGSNRYYADSVKG
CDRH3:
[SEQ ID NO: 119]
DRWNGMDV
CDRL1:
[SEQ ID NO: 120]
SGSSSNIGAGYDVH
CDRL2:
[SEQ ID NO: 121]
ANNQRPS
CDRL3:
[SEQ ID NO: 122]
AAWDDSLNGPWV
Antibody clone: 5H06
5H06-VH
[SEQ ID NO: 15]
EVQLLESGGGLVQPGGSLRLSCAASGFTFSSYGMHWVR-
QAPGKGLEWVAVISYD-
GSDTAYADSVKGRFTISRDNSKNTLYLQMNSLRAEDTAVY
YCARDHSVIGAFDIWGQGTLVTVSS
5H06-VL
[SEQ ID NO: 39]
QSVLTQPPSASGTPGQRVTISCSGSSSNIGSNTVNWYQQLPG
TAPKLLIYDNN-KRPSGVP-DRFSGSKSGT
SASLAISGLRSEDEADYYCSSYAGSNNVVFGGGTKLTVLG
CDR regions
CDRH1:
[SEQ ID NO: 123]
SYGMH
CDRH2:
[SEQ ID NO: 124]
VISYDGSDTAYADSVKG
CDRH3:
[SEQ ID NO: 125]
DHSVIGAFDI
CDRL1:
[SEQ ID NO: 126]
SGSSSNIGSNTVN
CDRL2:
[SEQ ID NO: 127]
DNNKRPS
CDRL3:
[SEQ ID NO: 128]
SSYAGSNNVV
Antibody clone: 6A09
6A09-VH
[SEQ ID NO: 16]
EVQLLESGGGLVQPGGSLRLSCAASGFTFSSYGMHWVRQAPGKGLEWVAV-
TSYDGNTKY-YANSVKGR
FTISRDNSKNTLYLQMNSLRAEDTAVYYCAREDCGGDCFDYWGQ
GTLVTVSS
6A09-VL
[SEQ ID NO: 40]
QSVLTQPPSASGTPGQRVTISCTGSSSNIGAGYDVHWYQQLPGTAPKLLI-
YGNSNRPSGVP-
DRFSGSKSGTSASLAISGLRSEDEADYYCAAWDDSLNEGVFGGGTKLTVLG
CDR regions
CDRH1:
[SEQ ID NO: 129]
SYGMH
CDRH2:
[SEQ ID NO: 130]
VTSYDGNTKYYANSVKG
CDRH3:
[SEQ ID NO: 131]
EDCGGDCFDY
CDRL1:
[SEQ ID NO: 132]
TGSSSNIGAGYDVH
CDRL2:
[SEQ ID NO: 133]
GNSNRPS
CDRL3:
[SEQ ID NO: 134]
AAWDDSLNEGV
Antibody clone: 6B01
6B01-VH
[SEQ ID NO: 17]
EVQLLESGGGLVQPGGSLRLSCAASGFTFSNYGMHWVR-
QAPGKGLEWVAVISYDGSNKY-YADSVKGRFT
ISRDNSKNTLYLQMNSLRAEDTAVYYCARDQLGEAFDIWGQGT
LVTVSS
6B01-VL
[SEQ ID NO: 41]
QSVLTQPPSASGTPGQRVTISCTGSSSNIGAGYDVHWYQQLPGTAPKLLI-
YDNNKRPSGVP-
DRFSGSKSGTSASLAISGLRSEDEADYYCATWDDSLSGPVFGGGTKLTVLG
CDR regions
CDRH1:
[SEQ ID NO: 135]
NYGMH
CDRH2:
[SEQ ID NO: 136]
VISYDGSNKYYADSVKG
CDRH3:
[SEQ ID NO: 137]
DQLGEAFDI
CDRL1:
[SEQ ID NO: 138]
TGSSSNIGAGYDVH
CDRL2:
[SEQ ID NO: 139]
DNNKRPS
CDRL3:
[SEQ ID NO: 140]
ATWDDSLSGPV
Antibody clone: 6C11
6C11-VH
[SEQ ID NO: 18]
EVQLLESGGGLVQPGGSLRLSCAASGFTFDDYGMSWVR-
QAPGKGLEWVSAISGSGSSTY-
YADSVKGRFTISRDNSKNTLYLQMNSLRAEDTAVYYCAGGDIDY
FDYWGQGTLVTVSS
6C11-VL
[SEQ ID NO: 42]
QSVLTQPPSASGTPGQRVTISCTGSSSNFGAGYDVHWYQQLPGTAPKLLI-
YENNKRPSGVP-
DRFSGSKSGTSASLAISGLRSEDEADYYCAAWDDSLNGPVFGGGTKLTVLG
CDR regions
CDRH1:
[SEQ ID NO: 141]
DYGMS
CDRH2:
[SEQ ID NO: 142]
AISGSGSSTYYADSVKG
CDRH3:
[SEQ ID NO: 143]
GDIDYFDY
CDRL1:
[SEQ ID NO: 144]
TGSSSNFGAGYDVH
CDRL2:
[SEQ ID NO: 145]
ENNKRPS
CDRL3:
[SEQ ID NO: 146]
AAWDDSLNGPV
Antibody clone: 6C12
6C12-VH
[SEQ ID NO: 19]
EVQLLESGGGLVQPGGSLRLSCAASGFTFSSYGMHWVR-
QAPGKGLEWVAVISYDGSNKY-
YADSVKGRFTISRDNSKNTLYLQMNSLRAEDTAVYYCARERRDAFD
IWGQGTLVTVSS
6C12-VL
[SEQ ID NO: 43]
QSVLTQPPSASGTPGQRVTISCTGSSSNIGAGYDVHWYQQLPGTAP-
KLLIYSDNQRPSGVP-
DRFSGSKSGTSASLAISGLRSEDEADYYCATWDSDTPVFGGGTKLTVLG
CDR regions
CDRH1:
[SEQ ID NO: 147]
SYGMH
CDRH2:
[SEQ ID NO: 148]
VISYDGSNKYYADSVKG
CDRH3:
[SEQ ID NO: 149]
ERRDAFDI
CDRL1:
[SEQ ID NO: 150]
TGSSSNIGAGYDVH
CDRL2:
[SEQ ID NO: 151]
SDNQRPS
CDRL3:
[SEQ ID NO: 152]
ATWDSDTPV
Antibody clone: 6D01
6D01-VH
[SEQ ID NO: 20]
EVQLLESGGGLVQPGGSLRLSCAASGFTFSSYGMHWVR-
QAPGKGLEWVAVISYDGSNKY-
YADSVKGRFTISRDNSKNTLYLQMNSLRAEDTAMYYCA
RDHSAAGYFDYWGQGTLVTVSS
6D01-VL
[SEQ ID NO: 44]
QSVLTQPPSASGTPGQRVTISCSGSSSNIGSNTVNWYQQLPGTAPKLLI-
YGNSIRPSGG-PDRFSG
SKSGTSASLAISGLRSEDEADYYCASWDDSLSSPVFGGGTKLTVLG
CDR regions
CDRH1:
[SEQ ID NO: 153]
SYGMH
CDRH2:
[SEQ ID NO: 154]
VISYDGSNKYYADSVKG
CDRH3:
[SEQ ID NO: 155]
DHSAAGYFDY
CDRL1:
[SEQ ID NO: 156]
SGSSSNIGSNTVN
CDRL2:
[SEQ ID NO: 157]
GNSIRPS
CDRL3:
[SEQ ID NO: 158]
ASWDDSLSSPV
Antibody clone: 6G03
6G03-VH
[SEQ ID NO: 21]
EVQLLESGGGLVQPGGSLRLSCAASGFTFGSYGMHWVR-
QAPGKGLEWVSGISWDSAI-
IDYAGSVKGRFTISRDNSKNTLYLQMNSLRAEDTAVYYCAKDE
AAAGAFDIWGQGTLVTVSS
6G03-VL
[SEQ ID NO: 45]
QSVLTQPPSASGTPGQRVTISCTGSSSNIGAGYDVHWYQQLPGTAPKLLI-
YGNTDRPSGVP-
DRFSGSKSGTSASLAISGLRSEDEADYYCAAWDDSLSGPVVFGGGTK
LTVLG
CDR regions
CDRH1:
[SEQ ID NO: 159]
SYGMH
CDRH2:
[SEQ ID NO: 160]
GISWDSAIIDYAGSVKG
CDRH3:
[SEQ ID NO: 161]
DEAAAGAFDI
CDRL1:
[SEQ ID NO: 162]
TGSSSNIGAGYDVH
CDRL2:
[SEQ ID NO: 163]
GNTDRPS
CDRL3:
[SEQ ID NO: 164]
AAWDDSLSGPVV
Antibody clone: 6G08
6G08-VH
[SEQ ID NO: 22]
EVQLLESGGGLVQPGGSLRLSCAASGFTLSSYGISWVRQAPGKG
LEWVSGIS-GSGGNTY-YADSVKGRFTISRDN
SKNTLYLQMNSLRAEDTAVYYCASSVGAYANDAFDIWG
QGTLVTVSS
6G08-VL
[SEQ ID NO: 46]
QSVLTQPPSASGTPGQRVTISCTGSSSNIGAGYDVHWYQQL
PGTAPKLLIYG-DTNRPSGVP-DRFSGSKSGTS
ASLAISGLRSEDEADYYCAAWDDSLNGPVFGGGTKLTVLG
CDR regions
CDRH1:
[SEQ ID NO: 165]
SYGIS
CDRH2:
[SEQ ID NO: 166]
GISGSGGNTYYADSVKG
CDRH3:
[SEQ ID NO: 167]
SVGAYANDAFDI
CDRL1:
[SEQ ID NO: 168]
TGSSSNIGAGYDVH
CDRL2:
[SEQ ID NO: 169]
GDTNRPS
CDRL3:
[SEQ ID NO: 170]
AAWDDSLNGPV
Antibody clone: 6G11
6G11-VH
[SEQ ID NO: 23]
EVQLLESGGGLVQPGGSLRLSCAASGFTFSSYGMHWVRQAPGKGLEW-
MAVISYDGSNKY-YADSVKGRFT
ISRDNSKNTLYLQMNSLRAEDTAVYYCARELYDAFDIWGQGTLV
TVSS
6G11-VL
[SEQ ID NO: 47]
QSVLTQPPSASGTPGQRVTISCTGSSSNIGAGYDVHWYQQLPGT
APKLLI-YADDHRPSGVP-DRFSGSKSGTSASLAI
SGLRSEDEADYYCASWDDSQRAVIFGGGTKLTVLG
CDR regions
CDRH1:
[SEQ ID NO: 171]
SYGMH
CDRH2:
[SEQ ID NO: 172]
VISYDGSNKYYADSVKG
CDRH3:
[SEQ ID NO: 173]
ELYDAFDI
CDRL1:
[SEQ ID NO: 174]
TGSSSNIGAGYDVH
CDRL2:
[SEQ ID NO: 175]
ADDHRPS
CDRL3:
[SEQ ID NO: 176]
ASWDDSQRAVI
Antibody clone: 6H08
6H08-VH
[SEQ ID NO: 24]
EVQLLESGGGLVQPGGSLRLSCAASGFTFNNYGMHWVR-
QAPGKGLEWVAVISYDGSNKY-YADSVKGRF
TISKDNSKNTLYLQMNSLRAEDTAVYYCAREYKDAFDIWGQGTLV
TVSS
6H08-VL
[SEQ ID NO: 48]
QSVLTQPPSASGTPGQRVTISCTGSSSNIGSNTVNWYQQLPGTAP
KLLIYDNN-KRPSGVP-
DRFSGSKSGTSASLAISGLRSEDEADYYCQAWGTGIRVFGGGTKLTVLG
CDR regions
CDRH1:
[SEQ ID NO: 177]
NYGMH
CDRH2:
[SEQ ID NO: 178]
VISYDGSNKYYADSVKG
CDRH3:
[SEQ ID NO: 179]
EYKDAFDI
CDRL1:
[SEQ ID NO: 180]
TGSSSNIGSNTVN
CDRL2:
[SEQ ID NO: 181]
DNNKRPS
CDRL3:
[SEQ ID NO: 182]
QAWGTGIRV
Antibody clone: 7C07
7C07-VH
[SEQ ID NO: 25]
EVQLLESGGGLVQPGGSLRLSCAASGFTFSSYGMHWVR-
QAPGKGLEWVAVISYDGSNKY-
YADSVKGRFTISRDNSQNTLYLQMNSLRAEDTAVYYCAR
EFGYIILDYWGQGTLVTVSS
7C07-VL
[SEQ ID NO: 49]
QSVLTQPPSASGTPGQRVTISCSGSSSNIGSNTVNWYQQLPGTAPKLLI-
YRDYERPSGVP-DRFSGSKSGTSASLA
ISGLRSEDEADYYCMAWDDSLSGVVFGGGTKLTVLG
CDR regions
CDRH1:
[SEQ ID NO: 183]
SYGMH
CDRH2:
[SEQ ID NO: 184]
VISYDGSNKYYADSVKG
CDRH3:
[SEQ ID NO: 185]
EFGYIILDY
CDRL1:
[SEQ ID NO: 186]
SGSSSNIGSNTVN
CDRL2:
[SEQ ID NO: 187]
RDYERPS
CDRL3:
[SEQ ID NO: 188]
MAWDDSLSGVV
Antibody clone: 4B02
4B02-VH
[SEQ ID NO: 26]
EVQLLESGGGLVQPGGSLRLSCAASGFTFSNHGMHWVR-
QAPGKGLEWVAVISYDGTNKY-YADSVRGRFTISRDN
SKNTLYLQMNSLRAEDTAVYYCARETWDAFDVWGQGT
LVTVSS
4B02-VL
[SEQ ID NO: 50]
QSVLTQPPSASGTPGQRVTISCSGSSSNIGSNNANWYQQLPGTA
PKLLIYDNN-KRPSGVP-
DRFSGSKSGTSASLAISGLRSEDEADYYCQAWDSSTVVFGGGTKLTVLG
CDR regions
CDRH1:
[SEQ ID NO: 189]
NHGMH
CDRH2:
[SEQ ID NO: 190]
VISYDGTNKYYADSVRG
CDRH3:
[SEQ ID NO: 191]
ETWDAFDV
CDRL1:
[SEQ ID NO: 192]
SGSSSNIGSNNAN
CDRL2:
[SEQ ID NO: 193]
DNNKRPS
CDRL3:
[SEQ ID NO: 194]
QAWDSSTVV

In some embodiments, which are sometimes preferred embodiments, the antibody molecule that specifically binds FcγRIIB comprises the following CDR regions: SEQ ID NO: 171 (CDRH1), SEQ ID NO: 172 (CDRH2), SEQ ID NO: 173 (CDRH3), SEQ ID NO: 174 (CDRL1), SEQ ID NO: 175 (CDRL2) and SEQ ID NO: 176 (CDRL3), i.e. the CDR regions of clone 6G11.

In some embodiments, which are sometimes preferred embodiments, the antibody molecule that specifically binds FcγRIIB comprises the following constant regions: SEQ ID NO: 1 (CH) and SEQ ID NO: 2 (CL) and the following variable regions: SEQ ID NO: 23 (VL) and SEQ ID NO: 47 (VH) i.e. the constant and variable regions of clone 6G11, which antibody molecule has further been modified to have reduced binding to Fcγ receptors via its Fc region. In some embodiments, which are sometimes preferred embodiments, the antibody molecule that specifically binds FcγRIIB comprises the following constant regions: SEQ ID NO: 195 (CH) and SEQ ID NO: 2 (CL) and the following variable regions: SEQ ID NO: 23 (VL) and SEQ ID NO: 47 (VH) i.e. the constant and variable regions of clone 6G11 including the N297Q mutation.

In some embodiments, the antibody molecule that specifically binds to a receptor present on a tumor cell is a human antibody molecule or an antibody molecule of human origin. In some such embodiments, the human antibody molecule or antibody molecule of human origin is an IgG antibody. In some such embodiments the human antibody molecule or antibody molecule of human origin is an IgG1 or an IgG2 antibody.

In some embodiments, the antibody molecule that specifically binds to a receptor present on a tumor cell, antibody molecule that specifically binds to a receptor present on a tumor cell is a humanized antibody molecule.

In some embodiments the antibody molecule that specifically binds to a receptor present on a tumor cell is a chimeric antibody.

As mentioned above, the antibody molecule that specifically binds to a receptor present on a tumor cell must have the ability to engage FcγRs.

The combination of an antibody molecule that specifically binds FcγRIIB and an antibody molecule that specifically binds to a receptor present on a tumor cell can be used use in the treatment of cancer.

“Patient” as the term is used herein refers to an animal, including human, that has been diagnosed as having an FcγRIIB negative cancer or as having a cancer that is considered as likely to be FcγRIIB negative cancer and/or that exhibits symptoms of such a cancer.

We include that the patient could be mammalian or non-mammalian. Preferably, the patient is a human or is a mammalian, such as a horse, or a cow, or a sheep, or a pig, or a camel, or a dog, or a cat. Most preferably, the mammalian patient is a human.

By “exhibit”, we include that the subject displays a cancer symptom and/or a cancer diagnostic marker, and/or the cancer symptom and/or a cancer diagnostic marker can be measured, and/or assessed, and/or quantified.

It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

Cancer treatments are often administered as a course of treatment, which is to say that the therapeutic agent is administered over a period of time. The length of time of the course of treatment will depend on a number of factors, which could include the type of therapeutic agent being administered, the type of cancer being treated, the severity of the cancer being treated, and the age and health of the patient, amongst others reasons.

By “during the treatment”, we include that the patient is currently receiving a course of treatment, and/or receiving a therapeutic agent, and/or receiving a course of a therapeutic agent.

In some embodiments of the present invention, the FcγRIIB negative cancer to be treated is a solid cancer.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11q, and/or deletions in the long arm of chromosome 6, and/or the deletion of 6q, and/or deletions in the short arm of chromosome 17, and/or the deletion of 17p, and/or the t(11:14) translocation, and/or the (q13:q32) translocation, and/or antigen gene receptor rearrangements, and/or BCL2 rearrangements, and/or BCL6 rearrangements, and/or t(14:18) translocations, and/or t(11:14) translocations, and/or (q13:q32) translocations, and/or (3:v) translocations, and/or (8:14) translocations, and/or (8:v) translocations, and/or t(11:14) and (q13:q32) translocations.

It is known that patients with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

In some embodiments, the target that the antibody molecule that specifically binds to a receptor present on a tumor cell binds to is human epidermal growth factor receptor 2 (HER2). In such embodiments, the FcγRIIB-negative cancer to be treated may be a cancer selected from the group consisting of breast cancers and gastric cancers.

In this context, breast cancers include metastatic breast cancer (MBC) and early breast cancer (EBC).

In this context, gastric cancers may also be denoted gastric adenocarcinomas or stomach cancers, and includes gastroesophageal junction (GEJ) adenocarcinoma. It further includes metastatic gastric cancer (MGC) and metastatic GEJ adenocarcinoma.

Trastuzumab (Herceptin®) is currently used, alone or in combination with chemotherapy or other drugs, for treatment of breast cancers expressing HER2, and such treatment has significantly improved overall survival. However, many patients remain uncured. Other patients develop trastuzumab resistance resulting in relapse of the disease, and in addition it has been shown that some breast cancers that are HER2-positive can become HER2-negative or low expression HER2 over time. Means of improving anti-HER2 therapy and overcoming resistance are therefore highly desirable in order to cure more patients.

In some embodiments of the invention, the FcγRIIB-negative cancer to be treated is a cancer with a low expression of HER2. A patient having cancer with low expression of HER2, often does not respond or respond well to standard of care treatment, such as treatment with trastuzumab and/or a trastuzumab biosimilar. However, by combining with an antibody molecule that specifically binds FcγRIIB via its Fab region, and that lacks an Fc region or has reduced binding to Fcγ receptors via its Fc region, as described herein, treatment of cancers with a low expression of HER2 becomes possible.

To determine if a cancer has a low expression of HER2, it is possible to use standard HER2 assays, such as an assay using immunohistochemistry (IHC) or fluorescence in situ hybridization (FISH), often performed on a biopsy taken from the patient.

An IHC test is based on to staining of the HER2 protein. When used to determine the amount of HER2 on the surface of cells in a breast cancer tissue sample, it gives a score of 0 to 3+. If the score is 0 to 1+, it's considered HER2-negative. If the score is 2+, it's considered borderline. A score of 3+ is considered HER2-positive. In this context, an IHC score for a patient with breast cancer of from 0 to 1+ may classify patient as having a cancer with a low expression of HER2. In some embodiments a score of 0 to 1+ is considered representing a HER2 low expressing cancer. In some embodiments of the present invention, a score of 3+ is considered representing a HER2 high expressing cancer, i.e. not a HER2 low expressing cancer. In the examples below, the well-established HER2 expressing immune competent Balb/C TUBO breast cancer tumor model was used. This model was adapted to allow for assessment of the ability of Fc:FcγR-impaired (Fc mute) anti-FcγRIIB antibodies to enhance anti-HER2 antitumor activity against cancer having a low expression of HER2 (HER2 low), and for comparison, also against cancer having ha high expression of HER2 (HER2 high). Accordingly, in the HER2 high expressing tumor model, animals received a full therapeutic dose of anti-HER2 antibody, which resulted in strong occupancy of tumor expressed HER2. In the HER2 low expressing model, animals received a lower dose of antibody resulting in approximately 10-fold fewer HER2 receptors on cancer cells being targeted by anti-HER2 antibody, as demonstrated by flow-cytometry analyses of tumors harvested from mice treated with fluorochrome conjugated anti-HER2 antibody. In this way, all other factors other than antibody-targeted HER2 receptors were identical, making this tumor model system ideal to assess and demonstrate anti-FcγRIIB-mediated enhancement of anti-HER2 efficacy against HER2 high expressing cancers, and to assess and demonstrate anti-FcγRIIB-mediated enablement of therapeutically meaningful effects of anti-HER2 against HER2 low expressing cancers.

The FISH, based on HER2 labelling, is more accurate than IHC, but it is more expensive and takes longer to return results. This is why an IHC test is usually the first test done to see if a cancer is HER2-positive. With the FISH test, you get a score of either positive or negative (some hospitals call a negative test result “zero”).

The two tests can be combined, for example if the IHC test results are borderline, it may be combined with a FISH test to give a better bases to determine if the cancer is HER2-positive. For example, IHC can be used to Generally only cancers that test IHC 3+ or FISH positive respond to the standard of care treatment with drugs that target HER2.

When the target that the second antibody molecule binds to is HER2, the second antibody molecule may be trastuzumab (Herceptin®) or a trastuzumab biosimilar, such as trastuzumab-anns (Kanjinti®), trastuzumab-qyyp (Trazimera®) trastuzumab-pkrb (Herzuma®), trastuzumab-dttb (Ontruzant®), or trastuzumabdkst (Ogivri®). By trastuzumab biosimilar, we here mean an antibody molecule that is highly similar to and has no clinically meaningful differences from trastuzumab. Alternatively, the second antibody molecule may be a toxin-conjugated, enhanced, variant of trastuzumab or a trastuzumab biosimilar, such as fam-trastuzumab-deruxtecan-nxki (Enhertu®), or T-DM1 or ado-trastuzumab emtansine (Kadcyla®), or other FcγR-engaging anti-HER2 antibody drug-conjugates.

In other cases, the second antibody may be used together with a third antibody, which may be tumor direct targeting e.g. the anti-HER2 antibody pertuzumab, or immune modulatory, e.g. an anti-PD-1/PD-L1 antibody. Further, the second antibody can be an anti-HER2 antibody used in any anti-HER2 containing therapeutic regimen.

In some embodiments of the invention, the FcγRIIB-negative cancer to be treated is a cancer in a patient that previously successfully has been treated with trastuzumab and/or a trastuzumab biosimilar, but then has developed resistance to trastuzumab or the trastuzumab biosimilar and therefor no longer responds to such treatment. cancer with a low expression of HER2. Combination with an antibody molecule that specifically binds FcγRIIB via its Fab region, and that lacks an Fc region or has reduced binding to Fcγ receptors via its Fc region, as described herein, makes it possible to overcome such resistance.

In some embodiments, the target that the antibody molecule that specifically binds to a receptor present on a tumor cell binds to is human epidermal growth factor receptor (EGFR). In such embodiments, the FcγRIIB-negative cancer to be treated may be a cancer selected from the group consisting of head and neck cancers and colorectal cancers.

In this context, head and neck cancers include locally or regionally advanced squamous cell carcinoma of the head and neck, recurrent locoregional disease or metastatic squamous cell carcinoma of the head and neck and recurrent or metastatic squamous cell carcinoma of the head and neck.

In this context, colorectal cancers include K-Ras wild-type, EGFR-expressing, metastatic colorectal cancer.

When the target that the second antibody molecule binds to is EGFR, the second antibody molecule may be cetuximab (Erbitux®), or a cetuximab biosimilar. By cetuximab biosimilar, we here mean an antibody molecule that is highly similar to and has no clinically meaningful differences from cetuximab.

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above-mentioned cancer types would be known to those skilled in medicine.

In some embodiments, the antibody molecule that specifically binds FcγRIIB and the antibody molecule that specifically binds to a receptor present on a tumor cell are administered simultaneously to the patient, meaning that they are either administered together at one or separately very close in time to each other.

In some embodiments the antibody molecule that specifically binds FcγRIIB is administered to the patient prior to administration of the antibody molecule that specifically binds to a receptor present on a tumor cell. Such sequential administration may be achieved by temporal separation of the two antibodies. Alternatively, or in combination with the first option, the sequential administration may also be achieved by spatial separation of the two antibody molecules, by administration of the antibody molecule that specifically binds FcγRIIB in a way, such as intratumoral, so that it reaches the cancer prior to the antibody molecule that specifically binds to a receptor present on a tumor cell, which is then administered in a way, such as systemically, so that it reaches the cancer after the antibody molecule that specifically binds FcγRIIB.

In some embodiments the antibody molecule that specifically binds to a receptor present on a tumor cell is administered to the patient prior to administration of the antibody molecule that specifically binds FcγRIIB. Such sequential administration may be achieved as described above.

It would be known to the person skilled in medicine, that medicines can be modified with different additives, for example to change the rate in which the medicine is absorbed by the body; and can be modified in different forms, for example to allow for a particular administration route to the body.

Accordingly, we include that the composition, and/or antibody, and/or medicament of at least one embodiment of the invention may be combined with an excipient and/or a pharmaceutically acceptable carrier and/or a pharmaceutically acceptable diluent and/or an adjuvant.

We also include that the composition, and/or antibody, and/or medicament of at least one embodiment of the invention may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The composition, and/or antibody, and/or agent, and/or medicament of at least one embodiment of the invention may be presented in unit-dose or multi-dose containers, for example sealed ampoules and vials, and may be stored in a freeze-dried (i.e. lyophilized) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the antibody molecule that specifically binds FcγRIIB and/or the antibody molecule that specifically binds to a receptor present on a tumor cell will usually be from 1 mg/kg bodyweight of the patient to 20 mg/kg, or in some cases even up to 100 mg/kg administered in single or divided doses. Lower doses may be used in special circumstances, for example in combination with prolonged administration. The physician in any event will determine the actual dosage which will be most suitable for any individual patient, and it will vary with the age, weight and response of the particular patient. The above dosages are exemplary of the average case. There can, of course, be individual instances where higher or lower dosage ranges are merited, and such are within the scope of this disclosure.

Generally, in humans, oral or parenteral administration of the composition, and/or antibody, and/or agent, and/or medicament of at least one embodiment of the invention is the preferred route, parenteral administration being most commonly used for antibodies. For veterinary use, the composition, and/or antibody, and/or agent and/or medicament of at least one embodiment of the invention are administered as a suitably acceptable formulation in accordance with normal veterinary practice and the veterinary surgeon will determine the dosing regimen and route of administration which will be most appropriate for a particular animal. Thus, the present disclosure provides a pharmaceutical formulation comprising an amount of an antibody and/or agent of at least one embodiment of the invention effective to treat various conditions (as described above and further below). Preferably, the composition, and/or antibody, and/or agent, and/or medicament is adapted for delivery by a route selected from the group comprising: intravenous (IV); subcutaneous (SC), intramuscular (IM), or intratumoral.

In some embodiments, either the first antibody molecule or the second antibody or both may be administered through the use of plasmids or viruses. Such plasmids then comprise nucleotide sequences encoding either the first antibody molecule or the second antibody or both. In some embodiments, nucleotide sequences encoding parts of or the full sequences of either the first antibody molecule or the second antibody or both integrated in a cell or viral genome or in a viriome in a virus; such a cell or virus then act as a delivery vehicle for either the first antibody molecule or the second antibody or both (or a delivery vehicle for a nucleotide sequence encoding either the first antibody molecule or the second antibody or both). For example, in some embodiments, such a virus may be in the form of a therapeutic oncolytic virus comprising nucleotide sequences encoding at least one of the antibody molecules described herein. In some embodiments, such an oncolytic virus comprises nucleotide sequences encoding a full-length human IgG antibody. Oncolytic viruses are known to those skilled in the arts of medicine and virology.

The present disclosure also includes composition, and/or antibody, and/or agent, and/or medicament comprising pharmaceutically acceptable acid or base addition salts of the polypeptide binding moieties of at least one embodiment of the present invention. The acids which are used to prepare the pharmaceutically acceptable acid addition salts of the aforementioned base compounds useful in this at least one embodiment of the invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to at least one embodiment of the present invention. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The agents and/or polypeptide binding moieties of at least one embodiment of the invention may be lyophilized for storage and reconstituted in a suitable carrier prior to use. Any suitable lyophilization method (e.g. spray drying, cake drying) and/or reconstitution techniques can be employed. It will be appreciated by those skilled in the art that lyophilization and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilized (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilization) when re-hydrated.

### BRIEF DESCRIPTION OF THE DRAWINGS

In the examples below, reference is made to the following figures:

FIG. 1. FIG. 1A-B show survival curves. Female BalbC mice (n=12) were injected subcutaneously with TUBO cells (1×10⁶). Tumor growth was monitored (measured by a caliper) and when tumors reached approximately 7×7 mm, mice were randomized and treated as indicated twice weekly. Tumor growth was followed twice a week until they reached a predetermined size (ethical endpoint) when the mice were euthanized. FIG. 1A. HER2 high cancer model. The therapeutic effect of anti-HER2 (10 mg/kg) in combination with Fc null anti-FcγRIIB-NA (AT130-2NA) was compared to an isotype control antibody (FITC IgG2a) and to anti-HER2 (10 mg/kg) single treatment. The mice were dosed three times (with 2-3 days between doses). In this model of HER2 high cancer, combined treatment with anti-FcγRIIB-NA (AT130-2NA) delayed tumor growth and increased the number of complete responders in comparison with anti-HER2 single agent treatment. The study was repeated 3 times with comparable results and results from 1 representative experiment is shown in FIG. 1A. FIG. 1B HER2 low cancer model. This figure shows the therapeutic effect of anti-HER2 (1 mg/kg) in combination with Fc null anti-FcγRIIB-NA (AT130-2NA) compared to an isotype control antibody (FITC IgG2a) and to 1 mg/kg of anti-HER2 as single treatment. The mice were dosed three times (with 2-3 days between doses). In this model of HER2 low cancer, combined treatment with anti-FcγRIIB-NA (AT130-2NA) delayed tumor growth and increased number of complete responders in comparison with anti-HER2 treatment alone, making anti-HER2 treatment of HER2 low cancers as effective as (single agent anti-HER2) treatment of HER2 high cancer. In FIG. 1C, Targeted HER2 receptors in HER2 High and HER2 Low experimental cancer models. Tumors were established as described in A-B and mice (n=3) were treated with 1 mg/kg or 10 mg/kg of fluorochrome (AF647) labelled anti-HER2. The mice were dosed two times (with 2-3 days between doses) and then the mice were euthanized, and tumors were collected. Tumors were enzymatically digested and the fluorochrome (AF647) labelled anti-HER2 quantified by FACS. The tumors form mice in the HER2 High model (injected with 10 mg/kg anti-HER2) showed a 10-fold increase in targeted HER2 receptors compared to tumors from mice in the HER2 Low model (injected with 1 mg/kg anti-HER2).

FIG. 2. Survival curve. Female BalbC mice (n=12) were injected subcutaneously with TUBO cells (1×10⁶). Tumor growth was monitored (measured by a caliper) and when tumors reached approx. 7×7 mm, mice were randomized and treated with therapeutic mAb twice weekly. Tumor growth was followed twice a week until they reached a predetermined size (ethical endpoint) when the mice were euthanized. The therapeutic effect of anti-HER2 (1 mg/kg) in combination with Fc null anti-FcγRIIB-NA (AT130-2NA) was compared to anti-HER2 in combination with wildtype anti-FcγRIIB (AT130-2 wt), to an isotype control antibody (FITC IgG2a) and to 1 mg/kg of anti-HER2 as single treatment. The mice were dosed three times (with 2-3 days between doses). Anti-HER2 in combination with anti-FcγRIIB-NA (AT130-2NA) showed delayed tumor growth in comparison with anti-HER2 treatment alone. This delay in tumor growth was not seen when anti-HER2 was combined with wildtype anti-FcγRIIB (AT130-2 wt).

FIG. 3. Female BalbC mice were injected subcutaneously with TUBO cells (1×10⁶). Tumor growth was monitored (measured by a caliper) and when tumors reached approx. 7×7 mm, mice were randomized and treated with therapeutic mAb twice weekly. 24 h after 3 injections, at day 7-8 after treatment start, mice were culled, and tumors harvested. Tumor single cell suspensions were analyzed for immune cell content by FACS. Fc null anti-FcγRIIB-NA is named AT-130-2NA in the figure. The number of myeloid cells, in particularly CD11b+F4/80+/MHCII^{low} were significantly increased in the groups treated with the combination of anti-HER2 and anti-FcγRIIB-NA.

FIG. 4. Metastasis covered lung area. Female C57 mice were injected intravenously with B16 cells (5×10⁵). Four days after tumor cell injection mice were injected with antibodies (10 mg/kg i.p—isotype control, TA99, AT130-2-NA and the combination of TA99 and AT130-2-NA). The treatment was given 5 times with an interval of 2-3 days. Day 21 after start of treatment mice were culled and metastasis content in the lungs was quantified. A reduction in lung metastasis was seen with the TA99 alone however, the effect of TA99 was greatly increased when being combined with anti-FcγRIIB-NA (AT130-2NA). Anti-FcγRIIB-NA has no therapeutic effect as single therapy.

### EXAMPLES

Specific, non-limiting examples which embody certain aspects of embodiments of the invention will now be described. To allow for examining the effect of blockade of FcγRIIB in complex in vivo systems, two sets of surrogate antibodies have been used. The murine equivalent of the Fc competent antibody 6G11 is called AT130-2. To Fc mute a human antibody (hence to render the binding to FcγR's severely impaired or negligible), we have replaced the amino acid position 297 from a N to a Q. To Fc mute a murine antibody, the same position is replaced from and N to an Q. Hence, in a murine system we will refer to AT-130, while this patent application concerns the human counterpart 6G11. In short, the human 6G11 corresponds to the murine surrogate AT1302-2 (both Fc:FcγR proficient, herein also denoted Fc competent), while the 6G11-N297Q corresponds to the AT130-3-N297A (both Fc:FcγR-impaired, herein also denoted Fc mute). We have previously shown that these human and mouse Fc:FcγR proficient or Fc:FcγR-impaired anti-FcγRIIB antibodies are functionally and biochemically equivalent (WO 2019/138005 and WO 2021/009358).

A different way to Fc mute an antibody (and well known to those skilled in the art) would be to take away the Fc part and form a Fab or F(ab′)₂ fragment.

The anti-HER2 mAb used below is clone 7.16.4 (mIgG2a) obtained from BioXcell.

Surrogate Anti-Mouse FcγRIIB mAb AT130-3-N297A Improves the In Vivo Anti-Tumor Effect of Anti-HER2 mAb, and Enables Treatment of HER2 Low Expressing Cancers

### Therapeutic Effect in the TUBO Tumor Model (HER2 High Expressing Cancer Model)

To assess the in vivo anti-tumor effect of the anti-mouse FcγRIIB mAb AT130-3-N297A in combination with an anti-HER2 mAb, the combination was investigated in vivo in the TUBO tumor model as described below.

Mice were bred and maintained in facilities in Lund, Sweden, in accordance with applicable rules and guidelines, including those of the facilities and the Swedish Board of Agriculture. Six to eight weeks old female BalbC mice were supplied by Taconic (Bomholt, Denmark) and maintained in local animal facilities. TUBO cells (University of Turin) were grown in glutamax buffered RPMI, supplemented with 10% FCS. When cells were semi confluent they were detached with trypsin and resuspended in sterile PBS at 10×10⁶ cells/ml. Glutamax buffered RPMI (RPMI medium), FCS (fetal calf serum) and PBS (phospatebufere Sine) all were from Invitrogen, and were used below. Mice were s.c. injected with 100 μl cell suspension corresponding to 1×10⁶ cells/mouse. 12-13 days after injection mice were treated twice weekly with 10 mg/kg antibody i.p. (isotype control, anti-HER2, AT130-3-N297A and the combination of anti-HER2 and AT130-3-N297A) as indicated in figures. Tumors were measured two times/week until they reached a diameter of 15 mm, where after the mice were terminated.

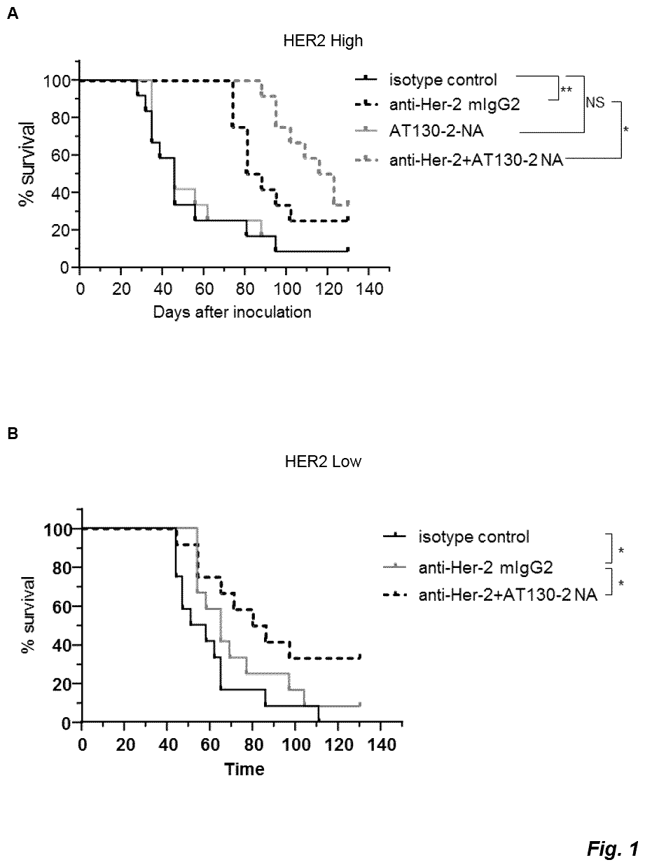

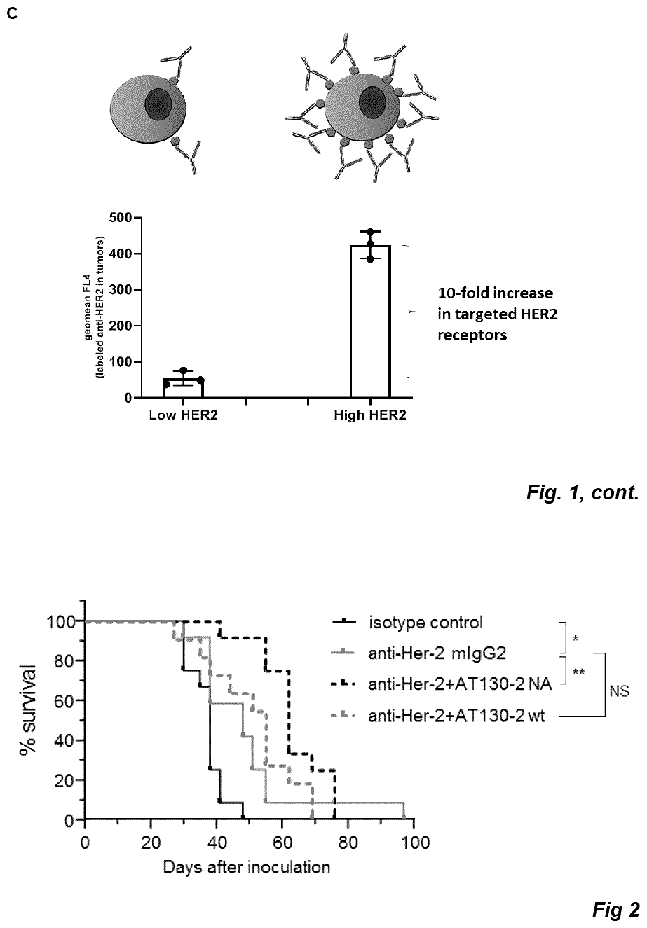

In this HER2 high experimental cancer model, anti-mouse FcγRIIB mAb AT130-3-N297A significantly improved anti-HER2 mediated survival compared to single agent anti-HER2 therapy (FIG. 1A).

### Anti-FcγRIIB Combination Treatment Enables Anti-HER2 Therapeutic Effects Against HER2 Low Expressing Cancers

To assess the in vivo effect of anti-mouse FcγRIIB mAb AT130-3-N297A in combination with anti-HER2 mAb against HER2 low cancers, the same HER2 high TUBO mouse tumor model described above, but using a lower dose of antibody resulting in fewer HER2 receptor on cancer cells being occupied and targeted by anti-HER2 antibody, was used. In this way, all other factors than antibody-targeted HER2 receptors, were identical, making this tumor model ideal to assess and demonstrate anti-FcγRIIB-enablement of anti-HER2 effects against HER2 low expressing cancers.

Mice were bred and maintained as above. Six to eight weeks old female BalbC mice were supplied by Taconic (Bomholt, Denmark) and maintained in local animal facilities. TUBO cells (University of Turin) were grown in glutamax buffered RPMI, supplemented with 10% FCS. When cells were semi confluent, they were detached with trypsin and resuspended in sterile PBS at 10×10⁶ cells/ml. Mice were s.c. injected with 100 μl cell suspension corresponding to 1×10⁶ cells/mouse. 12-13 days after injection mice were treated twice weekly with 1 mg/kg antibody i.p (isotype control, anti-HER2, and the combination of anti-HER2 and AT130-3-N297A) as indicated in figures. Tumors were measured two times/week until they reached a diameter of 15 mm, where after the mice were terminated

In this experimental HER2 low model, anti-HER2 single agent treatment showed severely impaired therapeutic efficacy, with only 1/10 treated animals cured compared to 3/10 mice cured in the HER2 high cancer model. Following combination with anti-mouse FcγRIIB mAb AT130-3-N297A, which by itself had no antitumor activity, full therapeutic efficacy similar to that observed in the HER2 high model (3/10 mice cured), was observed in the HER2 low tcancer model (FIG. 1B). Next, an experiment was designed to compare the amount of targeted HER2 in the HER2 high and HER2 low models. Tumors were established as described in A-B and mice (n=3) were treated with 1 mg/kg or 10 mg/kg of Alexa Flour (AF) 647 labelled anti-HER2, enabling determination of antibody-targeted HER2 in HER2 high and HER2 low cancer models. The mice were dosed two times (with 2-3 days between doses). Two days following the second injection, the mice were euthanized, and tumors were collected. Tumors were chopped into small pieces and enzymatically digested with a mixture of DNAse and Liberase at 37° C. Further the tumor solution was filtered through a cell strainer to obtain a single cell solution. The fluorochrome (AF647) labelled anti-HER2 in the tumors was quantified by FACS. Tumor cells from the HER2 high cancer model (mice injected with 10 mg/kg anti-HER2) showed a 10-fold increase in targeted HER2 receptors compared to tumor cells from the HER2 low cancer model (mice injected with 1 mg/kg anti-HER2).

No Therapeutic Effect when Fc Competent AT130-2 is Combined with Anti-HER2 mAb

To assess if the Fc competent AT130-2 also improves the in vivo anti-tumor effect of the anti-HER2 mAb, the combination was investigated in vivo in the tumor model as described below.

Mice were bred and maintained as above. Six to eight weeks old female BalbC mice were supplied by Taconic (Bomholt, Denmark) and maintained in local animal facilities. TUBO cells (University of Turin) were grown in glutamax buffered RPMI, supplemented with 10% FCS. When cells were semi confluent, they were detached with trypsin and resuspended in sterile PBS at 10×10⁶ cells/ml. Mice were s.c. injected with 100 μl cell suspension corresponding to 1×10⁶ cells/mouse. 12-13 days after injection mice were treated twice weekly with 10 mg/kg antibody i.p (isotype control, anti-HER2, or the combination of anti-HER2 with AT130-2-N297A or AT130-2 wt) as indicated in figures. Tumors were measured two times/week until they reached a diameter of 15 mm, where after the mice were terminated.

The Fc:FcγR-proficient (wt) AT130-2 thus shows no improved therapeutic anti-tumor effect when being combined with anti-HER2 (FIG. 2).

Improved Therapeutic Effect when Anti-HER2 Therapy is Combined with Antimouse FcγRIIB mAb AT130-3-N297A is Associated with Increased Influx of Myeloid Cells in Tumors.

To assess the mode of action of the therapeutic effect when anti-HER2 therapy is combined with anti-mouse FcγRIIB mAb AT130-2-N297A, immune profiling of treated tumors was made as described below.

Mice were bred and maintained as above. Six to eight weeks old female BalbC mice were supplied by Taconic (Bomholt, Denmark) and maintained in local animal facilities. TUBO cells (University of Turin) were grown in glutamax buffered RPMI, supplemented with 10% FCS. When cells were semi confluent, they were detached with trypsin and resuspended in sterile PBS at 10×10⁶ cells/ml. Mice were s.c. injected with 100 μl cell suspension corresponding to 1×10⁶ cells/mouse. Mice were injected with antibodies (10 mg/kg i.p—isotype control, anti-HER2, AT130-2-N297A and the combination of anti-HER2 and AT130-2-N297A) once the tumors reached a size of approximately 7×7 mm. 24 hours after 3 injections, at day 7-8 after treatment start (at this time point the tumors in the combination groups were in clear regression), tumors were harvested.

Tumors were chopped into small pieces and enzymatically digested with a mixture of DNAse and Liberase at 37° C. The tumor solution was filtered through a cell strainer to obtain single cell solution. The cell solution was blocked with IVIg (human normal immunoglobulin for intravascular administration, Kiovig, Takeda) prior to staining. Immune cells were identified and quantified by FACS using following markers: CD45, CD3, CD4, CD8, CD25, CD11b, Ly6C, Ly6G, MHCII, F4/80, CD49b and NK 1.1 (all from BD Biosciences).

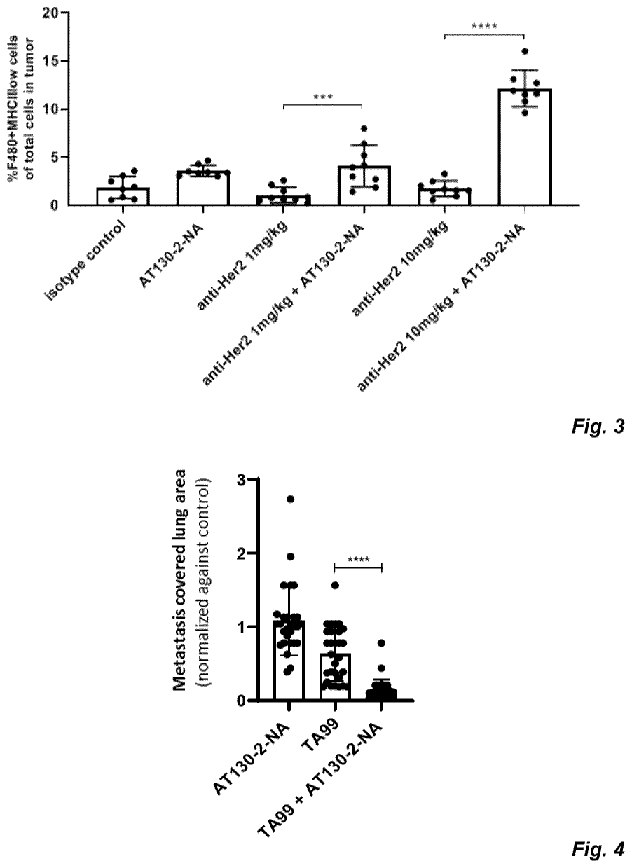

As seen in FIG. 3 the combination anti-HER2/anti-FcγRIIB-NA alters immune cell composition in tumors. The combined treatment of anti-HER2 and anti-FcγRIIB-NA results in an increased CD11b+/F4/80+ population compared to single treatment, consistent with increased recruitment of effector cells, and increased antibody-mediated depletion of HER2-targeted tumor cells. This increase is most profound in the HER2 high model (mice dosed with 10 mg/kg anti-HER2 dose) (FIG. 3).

### B16 Lung Metastasis Model

To assess the if anti-FcγRIIB-NA could enhance the depleting activity and the therapeutic efficacy of other tumor direct-targeting therapeutic antibodies targeting solid tumors, we investigated the therapeutic effect of combining anti-FcγRIIB-NA with TA99, an antibody specific to the gp75 melanoma tumor antigen, in the B16 metastatic melanoma model.

Mice were bred and maintained as above. Six to eight weeks old female C57 mice were supplied by Taconic (Bomholt, Denmark) and maintained in local animal facilities. B16 cells (ATCC) were grown in glutamax buffered RPMI, supplemented with 10% FCS. When cells were semi confluent, they were detached with trypsin and resuspended in sterile PBS at 2.5×10⁶ cells/ml. Mice were injected i.v with 200 μl cell suspension corresponding to 5×10⁵ cells/mouse. Four days after tumor cell injection mice were injected with antibodies (10 mg/kg i.p—isotype control, TA99, AT130-2-N297A and the combination of TA99 and AT130-2-N297A). The treatment was given 5 times with an interval of 2-3 days. Day 21 after start of treatment mice were culled and metastasis content in the lungs was quantified. FIG. 4

A moderate reduction in lung metastasis was observed following treatment with TA99 alone compared to untreated animals (FIG. 4). Following combined treatment with anti-FcγRIIB-NA, which by itself had no effect on metastasis formation, the therapeutic effect of the tumor direct-targeting antibody TA99 was greatly enhanced, significantly decreasing lung metastasis compared to TA99 single agent treatment. Thus, combination treatment with anti-FcγRIIB-NA enhances therapeutic efficacy of different tumor direct-targeting antibodies, specific to different tumor antigens relevant to different FcγRIIB− solid cancers.

1. (canceled)

2. A pharmaceutical composition comprising:
(i) a first antibody molecule that specifically binds FcγRIIB via its Fab region and that lacks an Fc region or has reduced binding to Fcγ receptors via the Fc region, and
(ii) a second antibody molecule that specifically binds to a receptor present on a tumor cell, wherein the second antibody molecule has an Fc region that binds to at least one activating Fcγ receptor;
wherein the pharmaceutical composition is used to treat an FcγRIIB-negative cancer in a patient.

3. A kit for treating an FcγRIIB-negative cancer comprising:
(i) a first antibody molecule that specifically binds FcγRIIB via its Fab region and that lacks an Fc region or has a reduced binding to Fcγ receptors via the Fc region, and
(ii) a second antibody molecule that specifically binds to a receptor present on a tumor cell, wherein the second antibody molecule has an Fc region that binds to at least one activating Fcγ receptor.

4. (canceled)

5. A method comprising:
administering, to treat an FcγRIIB-negative cancer in a patient:
(i) a first antibody molecule that specifically binds FcγRIIB via its Fab region and that lacks an Fc region or has reduced binding to Fcγ receptors via the Fc region, and
(ii) a second antibody molecule that specifically binds to a receptor present on a tumor cell, wherein the second antibody molecule has an Fc region that is capable of activating at least one activating Fcγ receptor.

6. The method according to claim 5, wherein the FcγRIIB-negative cancer is a solid cancer.

7. The method according to claim 5, wherein the binding of the second antibody molecule to the receptor on the tumor cell causes depletion of the tumor cell.

8. (canceled)

9. The method according to claim 5, wherein the second antibody molecule binds to human epidermal growth factor receptor 2 (HER2).

10. The method according to claim 9, wherein the FcγRIIB-negative cancer is selected from the group consisting of: breast cancer, gastric cancer, and combinations thereof.

11. The method according to claim 9, wherein the FcγRIIB-negative cancer has a low expression of HER2.

12. The method according to claim 9, wherein the FcγRIIB-negative cancer in the patient has previously been treated with an antibody molecule which specifically binds to HER2 but has developed resistance to the antibody molecule.

13. The method according to claim 9, wherein the second antibody molecule is selected from the group consisting of: trastuzumab, a trastuzumab biosimilar, and combinations thereof.

14. The method according to claim 5, wherein the second antibody molecule binds to human epidermal growth factor receptor (EGFR).

15. The method according to claim 14, wherein the cancer is selected from the group consisting of head and neck cancers, colorectal cancers, and combinations thereof.

16. The method according to claim 14, wherein the second antibody molecule is selected from the group consisting of: cetuximab, a cetuximab biosimilar, and combinations thereof.

17. The method according to claim 5, wherein the first antibody molecule is selected from the group consisting of a human antibody molecule, a humanized antibody molecule, an antibody molecule of human origin, a monoclonal antibody molecule, an antibody molecule of monoclonal origin, a full-length antibody, a chimeric antibody, a single chain antibody, a Fab fragment, a (Fab′)₂ fragment, a Fab′ fragment, a (Fab′)₂ fragment, a Fv fragment, and an scFv fragment, a human IgG antibody molecule having an aglycosylated Fc region, an IgG antibody molecule of human origin having an aglycosylated Fc region, and combinations thereof.

18. (canceled)

19. (canceled)

20. (canceled)

21. The method according to claim 17, wherein the IgG antibody molecule is selected from the group consisting of: an IgG1 antibody molecule, an IgG2 antibody molecule, an aglycoslated human IgG1 molecule, an aglycosylated humanized murine antibody, an aglycosylated humanized llama hcIgG antibody, an aglycosylated chimerized murine IgG, and combinations thereof.

22. (canceled)

23. The method according to claim 5, wherein the first antibody molecule has been aglycosylated through amino acid substitution in position 297.

24. The method according to claim 5, wherein the aglycosylation has occurred through an N297Q substitution.

25. The method according to claim 5, wherein the first antibody molecule comprises:
(i) a variable heavy chain (VH) comprising SEQ ID NO: 51, SEQ ID NO: 52, and SEQ ID NO: 53, and
a variable light chain (VL) comprising SEQ ID NO: 54, SEQ ID NO: 55, and SEQ ID NO: 56;
(ii) a VH comprising SEQ ID NO: 57, SEQ ID NO: 58, and SEQ ID NO: 59, and
a VL comprising SEQ ID NO: 60, SEQ ID NO: 61, and SEQ ID NO: 62;
(iii) a VH comprising SEQ ID NO: 63, SEQ ID NO: 64, and SEQ ID NO: 65, and
a VL comprising SEQ ID NO: 66, SEQ ID NO: 67, and SEQ ID NO: 68;
(iv) a VH comprising SEQ ID NO: 69, SEQ ID NO: 70, and SEQ ID NO: 71, and
a VL comprising SEQ ID NO: 72, SEQ ID NO: 73, and SEQ ID NO: 74;
(v) a VH comprising SEQ ID NO: 75, SEQ ID NO: 76, and SEQ ID NO: 77, and
a VL comprising SEQ ID NO: 78, SEQ ID NO: 79, and SEQ ID NO: 80;
(vi) a VH comprising SEQ ID NO: 81, SEQ ID NO: 82, and SEQ ID NO: 83, and
a VL comprising SEQ ID NO: 84, SEQ ID NO: 85, and SEQ ID NO: 86;
(vii) a VH comprising SEQ ID NO: 87, SEQ ID NO: 88, and SEQ ID NO: 89, and
a VL comprising SEQ ID NO: 90, SEQ ID NO: 91, and SEQ ID NO: 92;
(viii) a VH comprising SEQ ID NO: 93, SEQ ID NO: 94, and SEQ ID NO: 95, and
a VL comprising SEQ ID NO: 96, SEQ ID NO: 97, and SEQ ID NO: 98;
(ix) a VH comprising SEQ ID NO: 99, SEQ ID NO: 100, and SEQ ID NO: 101, and
a VL comprising SEQ ID NO: 102, SEQ ID NO: 103, and SEQ ID NO: 104;
(x) a VH comprising SEQ ID NO: 105% SEQ ID NO: 106, and SEQ ID NO: 107, and
a VL comprising SEQ ID NO: 108, SEQ ID NO: 109, and SEQ ID NO: 110;
(xi) a VH comprising SEQ ID NO: 111, SEQ ID NO: 112, and SEQ ID NO: 113, and
a VL comprising SEQ ID NO: 114, SEQ ID NO: 115, and SEQ ID NO: 116;
(xii) a VH comprising SEQ ID NO: 117, SEQ ID NO: 118, and SEQ ID NO: 119, and
a VL comprising SEQ ID NO: 120, SEQ ID NO: 121, and SEQ ID NO: 122;
(xiii) a VH comprising SEQ ID NO: 123, SEQ ID NO: 124, and SEQ ID NO: 125, and
a VL comprising SEQ ID NO: 126, SEQ ID NO: 127, and SEQ ID NO: 128;
(xiv) a VH comprising SEQ ID NO: 129, SEQ ID NO: 130, and SEQ ID NO: 131, and
a VL comprising SEQ ID NO: 132, SEQ ID NO: 133, and SEQ ID NO: 134;
(xv) a VH comprising SEQ ID NO: 135, SEQ ID NO: 136, and SEQ ID NO: 137, and
a VL comprising SEQ ID NO: 138, SEQ ID NO: 139, and SEQ ID NO: 140;
(xvi) a VH comprising SEQ ID NO: 141% SEQ ID NO: 142, and SEQ ID NO: 143, and
a VL comprising SEQ ID NO: 144, SEQ ID NO: 145, and SEQ ID NO: 146;
(xvii) a VH comprising SEQ ID NO: 147, SEQ ID NO: 148, and SEQ ID NO: 149, and
a VL comprising SEQ ID NO: 150, SEQ ID NO: 151, and SEQ ID NO: 152;
(xviii) a VH comprising SEQ ID NO: 153, SEQ ID NO: 154, and SEQ ID NO: 155, and
a VL comprising SEQ ID NO: 156, SEQ ID NO: 157, and SEQ ID NO: 158;
(xix) a VH comprising SEQ ID NO: 159, SEQ ID NO: 160, and SEQ ID NO: 161, and
a VL comprising SEQ ID NO: 162, SEQ ID NO: 163, and SEQ ID NO: 164;
(xx) a VH comprising SEQ ID NO: 165, SEQ ID NO: 166, and SEQ ID NO: 167, and
a VL comprising SEQ ID NO: 168, SEQ ID NO: 169, and SEQ ID NO: 170;
(xxi) a VH comprising SEQ ID NO: 171, SEQ ID NO: 172, and SEQ ID NO: 173, and
a VL comprising SEQ ID NO: 174, SEQ ID NO: 175, and SEQ ID NO: 176;
(xxii) a VH comprising SEQ ID NO: 177, SEQ ID NO: 178, and SEQ ID NO: 179, and
a VL comprising SEQ ID NO: 180, SEQ ID NO: 181, and SEQ ID NO: 182;
(xxiii) a VH comprising SEQ ID NO: 183, SEQ ID NO: 184, and SEQ ID NO: 185, and
a VL comprising SEQ ID NO: 186, SEQ ID NO: 187, and SEQ ID NO: 188; or
(xxiv) a VH comprising SEQ ID NO: 189, SEQ ID NO: 190, and SEQ ID NO: 191, and
a VL comprising SEQ ID NO: 192, SEQ ID NO: 193, and SEQ ID NO: 194.

26. The method according to claim 5, wherein the first antibody molecule comprises:
(i) a VH having SEQ ID NO: 3 and a VL having SEQ ID NO: 27;
(ii) a VH having SEQ ID NO: 4 and a VL having SEQ ID NO: 28;
(iii) a VH having SEQ ID NO: 5 and a VL having SEQ ID NO: 29;
(iv) a VH having SEQ ID NO: 6 and a VL having SEQ ID NO: 30;
(v) a VH having SEQ ID NO: 7 and a VL having SEQ ID NO: 31;
(vi) a VH having SEQ ID NO: 8 and a VL having SEQ ID NO: 32;
(vii) a VH having SEQ ID NO: 9 and a VL having SEQ ID NO:33;
(viii) a VH having SEQ ID NO: 10 and a VL having SEQ ID NO:34;
(ix) a VH having SEQ ID NO: 11 and a VL having SEQ ID NO:35;
(x) a VH having SEQ ID NO: 12 and a VL having SEQ ID NO:36;
(xi) a VH having SEQ ID NO: 13 and a VL having SEQ ID NO:37;
(xii) a VH having SEQ ID NO: 14 and a VL having SEQ ID NO:38;
(xiii) a VH having SEQ ID NO: 15 and a VL having SEQ ID NO:39;
(xiv) a VH having SEQ ID NO: 16 and a VL having SEQ ID NO:40;
(xv) a VH having SEQ ID NO: 17 and a VL having SEQ ID NO:41;
(xvi) a VH having SEQ ID NO: 18 and a VL having SEQ ID NO:42;
(xvii) a VH having SEQ ID NO: 19 and a VL having SEQ ID NO:43;
(xviii) a VH having SEQ ID NO: 20 and a VL having SEQ ID NO:44;
(xix) a VH having SEQ ID NO: 21 and a VL having SEQ ID NO:45;
(xx) a VH having SEQ ID NO: 22 and a VL having SEQ ID NO:46;
(xxi) a VH having SEQ ID NO: 23 and a VL having SEQ ID NO:47;
(xxii) a VH having SEQ ID NO: 24 and a VL having SEQ ID NO:48;
(xxiii) a VH having SEQ ID NO: 25 and a VL having SEQ ID NO:49; or
(xxiv) a VH having SEQ ID NO: 26 and a VL having SEQ ID NO:50.

27. The method according to claim 5,
wherein the first antibody molecule comprises:
a VH comprising SEQ ID NO: 171, SEQ ID NO: 172, and SEQ ID NO: 173, and
a VL comprising SEQ ID NO: 174, SEQ ID NO: 175, and SEQ ID NO: 176.

28. The method according to claim 5, wherein the first antibody molecule comprises a VH having SEQ ID NO: 23, and a VL having SEQ ID NO: 47.

29. The method according to claim 27, wherein the first antibody molecule has a constant heavy chain (CH) having SEQ ID NO: 195 and a constant light chain (CL) having SEQ ID NO: 2.

30. (canceled)

31. The kit according to claim 3, wherein the first antibody molecule has been aglycosylated through amino acid substitution in position 297.

32. The kit according to claim 31, wherein the aglycosylation has occurred through an N297Q substitution.


SEQUENCE LISTING (197 sequences)

SEQ ID NO: 1 - protein, 330 aa, Homo sapiens [sequence omitted]

SEQ ID NO: 2 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 118 aa, Homo sapiens
EVQLLESGGGLVQPGGSLRLSCAASGFTFSDYYMNWIRQTPGKGLEWVSLIGWDGGSTYYADSVKGRFTISRDNSENTLYLQMNSLRAEDTAVYYCARAYSGYELDYWGQGTLVTVSS

SEQ ID NO: 4 - protein, 117 aa, Homo sapiens
EVQLLESGGGLVQPGGSLRLSCAASGFTFSSYGMHWVRQAPGKGLEWVAFTRYDGSNKYYADSVRGRFTISRDNSKNTLYLQMNSLRAEDTAVYYCARENIDAFDVWGQGTLVTVSS

SEQ ID NO: 5 - protein, 122 aa, Homo sapiens
EVQLLESGGGLVQPGGSLRLSCAASGFTFSSYAMSWVRQAPGKGLEWVSSISDSGAGRYYADSVEGRFTISRDNSKNTLYLQMNSLRAEDTAVYYCARTHDSGELLDAFDIWGQGTLVTVSS

SEQ ID NO: 6 - protein, 124 aa, Homo sapiens
EVQLLESGGGLVQPGGSLRLSCAASGFTFSTYAMNWVRQVPGKGLEWVAVISYDGSNKNYVDSVKGRFTISRDNSKNTLYLQMNSLRAEDTAVYYCARNFDNSGYAIPDAFDIWGQGTLVTVSS

SEQ ID NO: 7 - protein, 121 aa, Homo sapiens
EVQLLESGGGLVQPGGSLRLSCAASGFTFSNAWMSWVRQAPGKGLEWVAYISRDADITHYPASVKGRFTISRDNSKNTLYLQMNSLRAEDTAVYYCTTGFDYAGDDAFDIWGQGTLVTVSS

SEQ ID NO: 8 - protein, 119 aa, Homo sapiens
EVQLLESGGGLVQPGGSLRLSCAASGFTFSDYYMSWVRQAPGKGLEWVALIGHDGNNKYYLDSLEGRFTISRDNSKNTLYLQMNSLRAEDTAVYYCARATDSGYDLLYWGQGTLVTVSS

SEQ ID NO: 9 - protein, 117 aa, Homo sapiens
EVQLLESGGGLVQPGGSLRLSCAASGFTFSDYYMSWIRQAPGKGLEWVSAIGFSDDNTYYADSVKGRFTISRDNSKNTLYLQMNSLRAEDTAVYYCAGGDGSGWSFWGQGTLVTVSS

SEQ ID NO: 10 - protein, 117 aa, Homo sapiens
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAREWRDAFDIWGQGTLVTVSS

SEQ ID NO: 11 - protein, 117 aa, Homo sapiens
EVQLLESGGGLVQPGGSLRLSCAASGFTFSTYGMHWVRQAPGKGLEWVAVISYDGSNKYYADSVKGRFTISRDNSKNTLYLQMNSLRAEDTAVYYCARENFDAFDVWGQGTLVTVSS

SEQ ID NO: 12 - protein, 117 aa, Homo sapiens
EVQLLESGGGLVQPGGSLRLSCAASGFTFSTYGMHWVRQAPGKGLEWVAVIAYDGSKKDYADSVKGRFTISRDNSKNTLYLQMNSLRAEDTAVYYCAREYRDAFDIWGQGTLVTVSS

SEQ ID NO: 13 - protein, 117 aa, Homo sapiens
EVQLLESGGGLVQPGGSLRLSCAASGFTFSSYGMHWVRQAPGKGLEWVAVISYDGINKDYADSMKGRFTISRDNSKNTLYLQMNSLRAEDTAVYYCARERKDAFDIWGQGTLVTVSS

SEQ ID NO: 14 - protein, 117 aa, Homo sapiens
EVQLLESGGGLVQPGGSLRLSCAASGFTFNNYGMHWVRQAPGKGLEWVAVISYDGSNRYYADSVKGRFTMSRDNSKNTLYLQMNSLRAEDTAVYYCARDRWNGMDVWGQGTLVTVSS

SEQ ID NO: 15 - protein, 119 aa, Homo sapiens
EVQLLESGGGLVQPGGSLRLSCAASGFTFSSYGMHWVRQAPGKGLEWVAVISYDGSDTAYADSVKGRFTISRDNSKNTLYLQMNSLRAEDTAVYYCARDHSVIGAFDIWGQGTLVTVSS

SEQ ID NO: 16 - protein, 119 aa, Homo sapiens
EVQLLESGGGLVQPGGSLRLSCAASGFTFSSYGMHWVRQAPGKGLEWVAVTSYDGNTKYYANSVKGRFTISRDNSKNTLYLQMNSLRAEDTAVYYCAREDCGGDCFDYWGQGTLVTVSS

SEQ ID NO: 17 - protein, 118 aa, Homo sapiens
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCARDQLGEAFDIWGQGTLVTVSS

SEQ ID NO: 18 - protein, 117 aa, Homo sapiens
EVQLLESGGGLVQPGGSLRLSCAASGFTFDDYGMSWVRQAPGKGLEWVSAISGSGSSTYYADSVKGRFTISRDNSKNTLYLQMNSLRAEDTAVYYCAGGDIDYFDYWGQGTLVTVSS

SEQ ID NO: 19 - protein, 117 aa, Homo sapiens
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARERRDAFDIWGQGTLVTVSS

SEQ ID NO: 20 - protein, 119 aa, Homo sapiens
EVQLLESGGGLVQPGGSLRLSCAASGFTFSSYGMHWVRQAPGKGLEWVAVISYDGSNKYYADSVKGRFTISRDNSKNTLYLQMNSLRAEDTAMYYCARDHSAAGYFDYWGQGTLVTVSS

SEQ ID NO: 21 - protein, 119 aa, Homo sapiens
EVQLLESGGGLVQPGGSLRLSCAASGFTFGSYGMHWVRQAPGKGLEWVSGISWDSAIIDYAGSVKGRFTISRDNSKNTLYLQMNSLRAEDTAVYYCAKDEAAAGAFDIWGQGTLVTVSS

SEQ ID NO: 22 - protein, 121 aa, Homo sapiens
EVQLLESGGGLVQPGGSLRLSCAASGFTLSSYGISWVRQAPGKGLEWVSGISGSGGNTYYADSVKGRFTISRDNSKNTLYLQMNSLRAEDTAVYYCASSVGAYANDAFDIWGQGTLVTVSS

SEQ ID NO: 23 - protein, 117 aa, Homo sapiens
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSS

SEQ ID NO: 24 - protein, 117 aa, Homo sapiens
EVQLLESGGGLVQPGGSLRLSCAASGFTFNNYGMHWVRQAPGKGLEWVAVISYDGSNKYYADSVKGRFTISKDNSKNTLYLQMNSLRAEDTAVYYCAREYKDAFDIWGQGTLVTVSS

SEQ ID NO: 25 - protein, 118 aa, Homo sapiens
EVQLLESGGGLVQPGGSLRLSCAASGFTFSSYGMHWVRQAPGKGLEWVAVISYDGSNKYYADSVKGRFTISRDNSQNTLYLQMNSLRAEDTAVYYCAREFGYIILDYWGQGTLVTVSS

SEQ ID NO: 26 - protein, 117 aa, Homo sapiens
EVQLLESGGGLVQPGGSLRLSCAASGFTFSNHGMHWVRQAPGKGLEWVAVISYDGTNKYYADSVRGRFTISRDNSKNTLYLQMNSLRAEDTAVYYCARETWDAFDVWGQGTLVTVSS

SEQ ID NO: 27 - protein, 111 aa, Homo sapiens
QSVLTQPPSASGTPGQRVTISCSGSSSNIGNNAVNWYQQLPGTAPKLLIYDNNNRPSGVPDRFSGSKSGTSASLAISGLRSEDEADYYCAAWDDSLNASIFGGGTKLTVLG

SEQ ID NO: 28 - protein, 111 aa, Homo sapiens
QSVLTQPPSASGTPGQRVTISCSGSSSNIGNNAVNWYQQLPGTAPKLLIYDNQQRPSGVPDRFSGSKSGTSASLAISGLRSEDEADYYCEAWDDRLFGPVFGGGTKLTVLG

SEQ ID NO: 29 - protein, 111 aa, Homo sapiens
QSVLTQPPSASGTPGQRVTISCSGSSSNIGSNHVLWYQQLPGTAPKLLIYGNSNRPSGVPDRFSGSKSGTSASLAISGLRSEDEADYYCAAWDDSLNGWVFGGGTKLTVLG

SEQ ID NO: 30 - protein, 112 aa, Homo sapiens
QSVLTQPPSASGTPGQRVTISCTGSSSNIGAGYDVHWYQQLPGTAPKLLIYDNNSRPSGVPDRFSGSKSGTSASLAISGLRSEDEADYYCAAWDDSLGGPVFGGGTKLTVLG

SEQ ID NO: 31 - protein, 112 aa, Homo sapiens
QSVLTQPPSASGTPGQRVTISCSGSSSNIGSNAVNWYQQLPGTAPKLLIYGNSDRPSGVPDRFSGSKSGTSASLAISGLRSEDEADYYCAAWDDSLNGRWVFGGGTKLTVLG

SEQ ID NO: 32 - protein, 111 aa, Homo sapiens
QSVLTQPPSASGTPGQRVTISCSGSSSNIGNNAVNWYQQLPGTAPKLLIYYDDLLPSGVPDRFSGSKSGTSASLAISGLRSEDEADYYCTTWDDSLSGVVFGGGTKLTVLG

SEQ ID NO: 33 - protein, 111 aa, Homo sapiens
QSVLTQPPSASGTPGQRVTISCSGSSSNIGNNAVNWYQQLPGTAPKLLIYDNNKRPSGVPDRFSGSKSGTSASLAISGLRSEDEADYYCATWDDSLRGWVFGGGTKLTVLG

SEQ ID NO: 34 - protein, 113 aa, Homo sapiens
QSVLTQPPSASGTPGQRVTISCTGSSSNIGAGYDVHWYQQLPGTAPKLLIYSDNQRPSGVPDRFSGSKSGTSASLAISGLRSEDEADYYCAAWDDSLSGSWVFGGGTKLTVLG

SEQ ID NO: 35 - protein, 113 aa, Homo sapiens
QSVLTQPPSASGTPGQRVTISCTGSSSNIGAGYDVHWYQQLPGTAPKLLIYSNSQRPSGVPDRFSGSKSGTSASLAISGLRSEDEADYYCAAWDDSLNGQVVFGGGTKLTVLG

SEQ ID NO: 36 - protein, 112 aa, Homo sapiens
QSVLTQPPSASGTPGQRVTISCTGSSSNIGAGYDVHWYQQLPGTAPKLLIYGNSNRPSGVPDRFSGSKSGTTASLAISGLRSEDEADYYCAAWDDSVSGWMFGGGTKLTVLG

SEQ ID NO: 37 - protein, 112 aa, Homo sapiens
QSVLTQPPSASGTPGQRVTISCTGSSSNIGAGYDVHWYQQLPGTAPKLLIYSNNQRPSGVPDRFSGSKSGTSASLAISGLRSEDEADYYCATWDDSLNGLVFGGGTKLTVLG

SEQ ID NO: 38 - protein, 113 aa, Homo sapiens
QSVLTQPPSASGTPGQRVTISCSGSSSNIGAGYDVHWYQQLPGTAPKLLIYANNQRPSGVPDRFSGSKSGTSASLAISGLRSEDEADYYCAAWDDSLNGPWVFGGGTKLTVLG

SEQ ID NO: 39 - protein, 110 aa, Homo sapiens
QSVLTQPPSASGTPGQRVTISCSGSSSNIGSNTVNWYQQLPGTAPKLLIYDNNKRPSGVPDRFSGSKSGTSASLAISGLRSEDEADYYCSSYAGSNNVVFGGGTKLTVLG

SEQ ID NO: 40 - protein, 112 aa, Homo sapiens
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLNEGVFGGGTKLTVLG

SEQ ID NO: 41 - protein, 112 aa, Homo sapiens
QSVLTQPPSASGTPGQRVTISCTGSSSNIGAGYDVHWYQQLPGTAPKLLIYDNNKRPSGVPDRFSGSKSGTSASLAISGLRSEDEADYYCATWDDSLSGPVFGGGTKLTVLG

SEQ ID NO: 42 - protein, 112 aa, Homo sapiens
QSVLTQPPSASGTPGQRVTISCTGSSSNFGAGYDVHWYQQLPGTAPKLLIYENNKRPSGVPDRFSGSKSGTSASLAISGLRSEDEADYYCAAWDDSLNGPVFGGGTKLTVLG

SEQ ID NO: 43 - protein, 110 aa, Homo sapiens
QSVLTQPPSASGTPGQRVTISCTGSSSNIGAGYDVHWYQQLPGTAPKLLIYSDNQRPSGVPDRFSGSKSGTSASLAISGLRSEDEADYYCATWDSDTPVFGGGTKLTVLG

SEQ ID NO: 44 - protein, 111 aa, Homo sapiens
QSVLTQPPSASGTPGQRVTISCSGSSSNIGSNTVNWYQQLPGTAPKLLIYGNSIRPSGGPDRFSGSKSGTSASLAISGLRSEDEADYYCASWDDSLSSPVFGGGTKLTVLG

SEQ ID NO: 45 - protein, 113 aa, Homo sapiens
QSVLTQPPSASGTPGQRVTISCTGSSSNIGAGYDVHWYQQLPGTAPKLLIYGNTDRPSGVPDRFSGSKSGTSASLAISGLRSEDEADYYCAAWDDSLSGPVVFGGGTKLTVLG

SEQ ID NO: 46 - protein, 112 aa, Homo sapiens
QSVLTQPPSASGTPGQRVTISCTGSSSNIGAGYDVHWYQQLPGTAPKLLIYGDTNRPSGVPDRFSGSKSGTSASLAISGLRSEDEADYYCAAWDDSLNGPVFGGGTKLTVLG

SEQ ID NO: 47 - protein, 112 aa, Homo sapiens
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLG

SEQ ID NO: 48 - protein, 109 aa, Homo sapiens
QSVLTQPPSASGTPGQRVTISCTGSSSNIGSNTVNWYQQLPGTAPKLLIYDNNKRPSGVPDRFSGSKSGTSASLAISGLRSEDEADYYCQAWGTGIRVFGGGTKLTVLG

SEQ ID NO: 49 - protein, 111 aa, Homo sapiens
QSVLTQPPSASGTPGQRVTISCSGSSSNIGSNTVNWYQQLPGTAPKLLIYRDYERPSGVPDRFSGSKSGTSASLAISGLRSEDEADYYCMAWDDSLSGVVFGGGTKLTVLG

SEQ ID NO: 50 - protein, 109 aa, Homo sapiens
QSVLTQPPSASGTPGQRVTISCSGSSSNIGSNNANWYQQLPGTAPKLLIYDNNKRPSGVPDRFSGSKSGTSASLAISGLRSEDEADYYCQAWDSSTVVFGGGTKLTVLG

SEQ ID NO: 51 - protein, 5 aa, Homo sapiens
DYYMN

SEQ ID NO: 52 - protein, 17 aa, Homo sapiens
LIGWDGGSTYYADSVKG

SEQ ID NO: 53 - protein, 9 aa, Homo sapiens
AYSGYELDY

SEQ ID NO: 54 - protein, 13 aa, Homo sapiens
SGSSSNIGNNAVN

SEQ ID NO: 55 - protein, 7 aa, Homo sapiens
DNNNRPS

SEQ ID NO: 56 - protein, 11 aa, Homo sapiens
AAWDDSLNASI

SEQ ID NO: 57 - protein, 5 aa, Homo sapiens
SYGMH

SEQ ID NO: 58 - protein, 17 aa, Homo sapiens
FTRYDGSNKYYADSVRG

SEQ ID NO: 59 - protein, 8 aa, Homo sapiens
ENIDAFDV

SEQ ID NO: 60 - protein, 13 aa, Homo sapiens
SGSSSNIGNNAVN

SEQ ID NO: 61 - protein, 7 aa, Homo sapiens
DNQQRPS

SEQ ID NO: 62 - protein, 9 aa, Homo sapiens
WDDRLFGPV

SEQ ID NO: 63 - protein, 5 aa, Homo sapiens
SYAMS

SEQ ID NO: 64 - protein, 17 aa, Homo sapiens
SISDSGAGRYYADSVEG

SEQ ID NO: 65 - protein, 13 aa, Homo sapiens
THDSGELLDAFDI

SEQ ID NO: 66 - protein, 13 aa, Homo sapiens
SGSSSNIGSNHVL

SEQ ID NO: 67 - protein, 7 aa, Homo sapiens
GNSNRPS

SEQ ID NO: 68 - protein, 11 aa, Homo sapiens
AAWDDSLNGWV

SEQ ID NO: 69 - protein, 5 aa, Homo sapiens
TYAMN

SEQ ID NO: 70 - protein, 17 aa, Homo sapiens
VISYDGSNKNYVDSVKG

SEQ ID NO: 71 - protein, 15 aa, Homo sapiens
NFDNSGYAIPDAFDI

SEQ ID NO: 72 - protein, 14 aa, Homo sapiens
TGSSSNIGAGYDVH

SEQ ID NO: 73 - protein, 7 aa, Homo sapiens
DNNSRPS

SEQ ID NO: 74 - protein, 11 aa, Homo sapiens
AAWDDSLGGPV

SEQ ID NO: 75 - protein, 5 aa, Homo sapiens
NAWMS

SEQ ID NO: 76 - protein, 17 aa, Homo sapiens
YISRDADITHYPASVKG

SEQ ID NO: 77 - protein, 12 aa, Homo sapiens
GFDYAGDDAFDI

SEQ ID NO: 78 - protein, 13 aa, Homo sapiens
SGSSSNIGSNAVN

SEQ ID NO: 79 - protein, 7 aa, Homo sapiens
GNSDRPS

SEQ ID NO: 80 - protein, 12 aa, Homo sapiens
AAWDDSLNGRWV

SEQ ID NO: 81 - protein, 5 aa, Homo sapiens
DYYMS

SEQ ID NO: 82 - protein, 17 aa, Homo sapiens
LIGHDGNNKYYLDSLEG

SEQ ID NO: 83 - protein, 10 aa, Homo sapiens
ATDSGYDLLY

SEQ ID NO: 84 - protein, 13 aa, Homo sapiens
SGSSSNIGNNAVN

SEQ ID NO: 85 - protein, 7 aa, Homo sapiens
YDDLLPS

SEQ ID NO: 86 - protein, 11 aa, Homo sapiens
TTWDDSLSGVV

SEQ ID NO: 87 - protein, 5 aa, Homo sapiens
DYYMS

SEQ ID NO: 88 - protein, 17 aa, Homo sapiens
AIGFSDDNTYYADSVKG

SEQ ID NO: 89 - protein, 8 aa, Homo sapiens
GDGSGWSF

SEQ ID NO: 90 - protein, 13 aa, Homo sapiens
SGSSSNIGNNAVN

SEQ ID NO: 91 - protein, 7 aa, Homo sapiens
DNNKRPS

SEQ ID NO: 92 - protein, 11 aa, Homo sapiens
ATWDDSLRGWV

SEQ ID NO: 93 - protein, 5 aa, Homo sapiens
NYGMH

SEQ ID NO: 94 - protein, 17 aa, Homo sapiens
VISYDGSNKYYADSVKG

SEQ ID NO: 95 - protein, 7 aa, Homo sapiens
WRDAFDI

SEQ ID NO: 96 - protein, 14 aa, Homo sapiens
TGSSSNIGAGYDVH

SEQ ID NO: 97 - protein, 7 aa, Homo sapiens
SDNQRPS

SEQ ID NO: 98 - protein, 12 aa, Homo sapiens
AAWDDSLSGSWV

SEQ ID NO: 99 - protein, 5 aa, Homo sapiens
TYGMH

SEQ ID NO: 100 - protein, 17 aa, Homo sapiens
VISYDGSNKYYADSVKG

SEQ ID NO: 101 - protein, 8 aa, Homo sapiens
ENFDAFDV

SEQ ID NO: 102 - protein, 14 aa, Homo sapiens
TGSSSNIGAGYDVH

SEQ ID NO: 103 - protein, 7 aa, Homo sapiens
SNSQRPS

SEQ ID NO: 104 - protein, 12 aa, Homo sapiens
AAWDDSLNGQVV

SEQ ID NO: 105 - protein, 5 aa, Homo sapiens
TYGMH

SEQ ID NO: 106 - protein, 17 aa, Homo sapiens
VIAYDGSKKDYADSVKG

SEQ ID NO: 107 - protein, 8 aa, Homo sapiens
EYRDAFDI

SEQ ID NO: 108 - protein, 14 aa, Homo sapiens
TGSSSNIGAGYDVH

SEQ ID NO: 109 - protein, 7 aa, Homo sapiens
GNSNRPS

SEQ ID NO: 110 - protein, 11 aa, Homo sapiens
AAWDDSVSGWM

SEQ ID NO: 111 - protein, 5 aa, Homo sapiens
SYGMH

SEQ ID NO: 112 - protein, 17 aa, Homo sapiens
VISYDGINKDYADSMKG

SEQ ID NO: 113 - protein, 8 aa, Homo sapiens
ERKDAFDI

SEQ ID NO: 114 - protein, 14 aa, Homo sapiens
TGSSSNIGAGYDVH

SEQ ID NO: 115 - protein, 7 aa, Homo sapiens
SNNQRPS

SEQ ID NO: 116 - protein, 11 aa, Homo sapiens
ATWDDSLNGLV

SEQ ID NO: 117 - protein, 5 aa, Homo sapiens
NYGMH

SEQ ID NO: 118 - protein, 17 aa, Homo sapiens
VISYDGSNRYYADSVKG

SEQ ID NO: 119 - protein, 8 aa, Homo sapiens
DRWNGMDV

SEQ ID NO: 120 - protein, 14 aa, Homo sapiens
SGSSSNIGAGYDVH

SEQ ID NO: 121 - protein, 7 aa, Homo sapiens
ANNQRPS

SEQ ID NO: 122 - protein, 12 aa, Homo sapiens
AAWDDSLNGPWV

SEQ ID NO: 123 - protein, 5 aa, Homo sapiens
SYGMH

SEQ ID NO: 124 - protein, 17 aa, Homo sapiens
VISYDGSDTAYADSVKG

SEQ ID NO: 125 - protein, 10 aa, Homo sapiens
DHSVIGAFDI

SEQ ID NO: 126 - protein, 13 aa, Homo sapiens
SGSSSNIGSNTVN

SEQ ID NO: 127 - protein, 7 aa, Homo sapiens
DNNKRPS

SEQ ID NO: 128 - protein, 10 aa, Homo sapiens
SSYAGSNNVV

SEQ ID NO: 129 - protein, 5 aa, Homo sapiens
SYGMH

SEQ ID NO: 130 - protein, 17 aa, Homo sapiens
VTSYDGNTKYYANSVKG

SEQ ID NO: 131 - protein, 10 aa, Homo sapiens
EDCGGDCFDY

SEQ ID NO: 132 - protein, 14 aa, Homo sapiens
TGSSSNIGAGYDVH

SEQ ID NO: 133 - protein, 7 aa, Homo sapiens
GNSNRPS

SEQ ID NO: 134 - protein, 11 aa, Homo sapiens
AAWDDSLNEGV

SEQ ID NO: 135 - protein, 5 aa, Homo sapiens
NYGMH

SEQ ID NO: 136 - protein, 17 aa, Homo sapiens
VISYDGSNKYYADSVKG

SEQ ID NO: 137 - protein, 9 aa, Homo sapiens
DQLGEAFDI

SEQ ID NO: 138 - protein, 14 aa, Homo sapiens
TGSSSNIGAGYDVH

SEQ ID NO: 139 - protein, 7 aa, Homo sapiens
DNNKRPS

SEQ ID NO: 140 - protein, 11 aa, Homo sapiens
ATWDDSLSGPV

SEQ ID NO: 141 - protein, 5 aa, Homo sapiens
DYGMS

SEQ ID NO: 142 - protein, 17 aa, Homo sapiens
AISGSGSSTYYADSVKG

SEQ ID NO: 143 - protein, 8 aa, Homo sapiens
GDIDYFDY

SEQ ID NO: 144 - protein, 14 aa, Homo sapiens
TGSSSNFGAGYDVH

SEQ ID NO: 145 - protein, 7 aa, Homo sapiens
ENNKRPS

SEQ ID NO: 146 - protein, 11 aa, Homo sapiens
AAWDDSLNGPV

SEQ ID NO: 147 - protein, 5 aa, Homo sapiens
SYGMH

SEQ ID NO: 148 - protein, 17 aa, Homo sapiens
VISYDGSNKYYADSVKG

SEQ ID NO: 149 - protein, 8 aa, Homo sapiens
ERRDAFDI

SEQ ID NO: 150 - protein, 14 aa, Homo sapiens
TGSSSNIGAGYDVH

SEQ ID NO: 151 - protein, 7 aa, Homo sapiens
SDNQRPS

SEQ ID NO: 152 - protein, 9 aa, Homo sapiens
ATWDSDTPV

SEQ ID NO: 153 - protein, 5 aa, Homo sapiens
SYGMH

SEQ ID NO: 154 - protein, 17 aa, Homo sapiens
VISYDGSNKYYADSVKG

SEQ ID NO: 155 - protein, 10 aa, Homo sapiens
DHSAAGYFDY

SEQ ID NO: 156 - protein, 13 aa, Homo sapiens
SGSSSNIGSNTVN

SEQ ID NO: 157 - protein, 7 aa, Homo sapiens
GNSIRPS

SEQ ID NO: 158 - protein, 11 aa, Homo sapiens
ASWDDSLSSPV

SEQ ID NO: 159 - protein, 5 aa, Homo sapiens
SYGMH

SEQ ID NO: 160 - protein, 17 aa, Homo sapiens
GISWDSAIIDYAGSVKG

SEQ ID NO: 161 - protein, 10 aa, Homo sapiens
DEAAAGAFDI

SEQ ID NO: 162 - protein, 14 aa, Homo sapiens
TGSSSNIGAGYDVH

SEQ ID NO: 163 - protein, 7 aa, Homo sapiens
GNTDRPS

SEQ ID NO: 164 - protein, 12 aa, Homo sapiens
AAWDDSLSGPVV

SEQ ID NO: 165 - protein, 5 aa, Homo sapiens
SYGIS

SEQ ID NO: 166 - protein, 17 aa, Homo sapiens
GISGSGGNTYYADSVKG

SEQ ID NO: 167 - protein, 12 aa, Homo sapiens
SVGAYANDAFDI

SEQ ID NO: 168 - protein, 14 aa, Homo sapiens
TGSSSNIGAGYDVH

SEQ ID NO: 169 - protein, 7 aa, Homo sapiens
GDTNRPS

SEQ ID NO: 170 - protein, 11 aa, Homo sapiens
AAWDDSLNGPV

SEQ ID NO: 171 - protein, 5 aa, Homo sapiens
SYGMH

SEQ ID NO: 172 - protein, 17 aa, Homo sapiens
VISYDGSNKYYADSVKG

SEQ ID NO: 173 - protein, 8 aa, Homo sapiens
ELYDAFDI

SEQ ID NO: 174 - protein, 14 aa, Homo sapiens
TGSSSNIGAGYDVH

SEQ ID NO: 175 - protein, 7 aa, Homo sapiens
ADDHRPS

SEQ ID NO: 176 - protein, 11 aa, Homo sapiens
ASWDDSQRAVI

SEQ ID NO: 177 - protein, 5 aa, Homo sapiens
NYGMH

SEQ ID NO: 178 - protein, 13 aa, Homo sapiens
VISYDGSNKYYAD

SEQ ID NO: 179 - protein, 8 aa, Homo sapiens
EYKDAFDI

SEQ ID NO: 180 - protein, 13 aa, Homo sapiens
TGSSSNIGSNTVN

SEQ ID NO: 181 - protein, 7 aa, Homo sapiens
DNNKRPS

SEQ ID NO: 182 - protein, 9 aa, Homo sapiens
QAWGTGIRV

SEQ ID NO: 183 - protein, 5 aa, Homo sapiens
SYGMH

SEQ ID NO: 184 - protein, 17 aa, Homo sapiens
VISYDGSNKYYADSVKG

SEQ ID NO: 185 - protein, 9 aa, Homo sapiens
EFGYIILDY

SEQ ID NO: 186 - protein, 13 aa, Homo sapiens
SGSSSNIGSNTVN

SEQ ID NO: 187 - protein, 7 aa, Homo sapiens
RDYERPS

SEQ ID NO: 188 - protein, 11 aa, Homo sapiens
MAWDDSLSGVV

SEQ ID NO: 189 - protein, 5 aa, Homo sapiens
NHGMH

SEQ ID NO: 190 - protein, 17 aa, Homo sapiens
VISYDGTNKYYADSVRG

SEQ ID NO: 191 - protein, 8 aa, Homo sapiens
ETWDAFDV

SEQ ID NO: 192 - protein, 13 aa, Homo sapiens
SGSSSNIGSNNAN

SEQ ID NO: 193 - protein, 7 aa, Homo sapiens
DNNKRPS

SEQ ID NO: 194 - protein, 9 aa, Homo sapiens
QAWDSSTVV

SEQ ID NO: 195 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 197 - protein, 105 aa, Mus musculus
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS